(19) United States
(12) Patent Application Publication
(10) Pub. No.: US 2025/0375589 A1
(43) Pub. Date: Dec. 11, 2025

(54) CATHETER ASSEMBLY

(71) Applicant: ConvaTec Limited, Flintshire (GB)

(72) Inventors: Michal Weber, Flintshire (GB); Aliaksandr Piashevich, Flintshire (GB); David Donnelly, Flintshire (GB)

(21) Appl. No.: 19/091,317

(22) Filed: Mar. 26, 2025

Related U.S. Application Data
(63) Continuation of application No. PCT/GB2023/052470, filed on Sep. 25, 2023.
(60) Provisional application No. 63/409,896, filed on Sep. 26, 2022.

(30) Foreign Application Priority Data
Nov. 9, 2022 (GB) 2216709.2

(51) Int. Cl. A61M 25/00

(52) U.S. Cl. CPC A61M 25/0023; A61M 25/0043

(57) ABSTRACT

A catheter assembly comprises a catheter, with a proximal end for insertion into the body and a distal end, and a housing. The housing comprises a base and a body rotatable with respect to one another to define a first configuration and a second configuration of the housing. In the first configuration the housing prevents passage of the proximal end of the catheter therethrough. In the second configuration the housing permits passage of the proximal end of the catheter therethrough. Preferably, the base and body comprise apertures to allow passage of the catheter therethrough. The apertures are preferably formed by tubes. Preferably, the apertures/tubes are misaligned in the first configuration and aligned in the second configuration. Preferably, the housing comprises a rotation guide to restrict linear movement of the base with respect to the body during rotation of the base with respect to the body.

### TECHNICAL FIELD OF THE INVENTION

The present invention relates to catheter assemblies. In particular the invention concerns urinary catheter assemblies, and most particularly, but not exclusively intermittent male urinary catheter assemblies, especially “closed” catheter assemblies.

### BACKGROUND TO THE INVENTION

A catheter is a medical device comprising a hollow catheter tube designed for insertion into canals, vessels, passageways or body cavities to permit injection, drainage or withdrawal of fluids or substances therefrom, or to ensure said canals, vessels, passageways etc. remain open. Urinary catheters are designed for use for insertion into a user's bladder via the urethra to drain the bladder.

To maximise comfort and minimise the risk of trauma and/or infection, an outer surface of the catheter tube is typically wetted using a wetting agent prior to insertion by the user. In further developments, the catheter tube itself comprises, is integrated with or is coated with a hydrophilic component (e.g. a hydrophilic polymer) which serves to reduce friction further upon application of the wetting agent.

Some catheters may be supplied pre-wetted in a packaging, for instance, where the catheter is at least partially submerged within wetting agent within the packaging. Whilst this may ensure the catheter tube is adequately wetted prior to use, such arrangements suffer in that components of the catheter other than the catheter tube such as a gripper element or funnel can also become wetted. This has a detrimental effect of the experience of the user where it may become difficult to hold and direct the catheter tube as required. This is particularly problematic where the user is performing self-catheterisation. Further, having the catheter submerged may effectively reduce the shelf-life of the catheter due to long-term exposure of components of the catheter to moisture.

It is therefore seen advantageous to provide a catheter which may be wetted at or immediately prior to the point of use.

In an attempt to address this, some catheters are provided in packaging which includes a rupturable container or sachet within the packaging which a user may burst to release the wetting agent. Typically, this involves the user squeezing the packaging to cause the container/sachet to break. However, such arrangements experience similar problems to those discussed above where the wetting agent is allowed to come into contact with other components of the catheter. Such arrangements also result in the possibility of the catheter tube not being fully wetted, or indeed wetted at all, prior to use. This can be harmful for the user. Furthermore such systems may require a degree of dexterity and offer no feedback to ensure wetting has occurred.

It is therefore advantageous to provide a catheter which includes a means of easily supplying a wetting agent solely to the catheter tube to improve user experience.

Manual dexterity can also be a problem when opening packaging in order to access the catheter as it can lead the user to incorrectly opening packaging, for example by tearing, which can result in the catheter coming into contact with dirt and being rendered unsafe for use. It is therefore advantageous to provide a catheter in packaging that is may be easily opened by the user while maintaining the catheter in a clean and usable state.

It is an aim of an embodiment or embodiments of the invention to overcome or at least partially mitigate one or more problems with the prior art and/or to provide an improved intermittent catheter.

### SUMMARY OF THE INVENTION

The invention concerns a catheter assembly. The assembly may comprise a catheter comprising a proximal end for insertion into the body and a distal end, and a wetting mechanism. The wetting mechanism may be arranged at the proximal end of the catheter. The wetting mechanism may comprise a base and a body. The body may comprise a fluid reservoir. The base and body may be rotatable with respect to one another to define a first configuration and a second configuration of the wetting mechanism. In the second configuration the wetting mechanism may permit release of fluid from the fluid reservoir to wet the catheter.

According to a broad aspect, there is provided a catheter assembly comprising: a catheter comprising a proximal end for insertion into the body and a distal end, and a wetting mechanism arranged at the proximal end of the catheter, wherein the wetting mechanism comprises a base and a body, the body comprising a fluid reservoir, the base and body being rotatable with respect to one another to define a first configuration and a second configuration of the wetting mechanism, wherein in the second configuration the wetting mechanism permits release of fluid from the fluid reservoir to wet the catheter.

The catheter assembly may comprise a sleeve configured to enclose the catheter. In the second configuration the wetting mechanism may permit release of fluid from the fluid reservoir into the sleeve to wet the catheter

According to a first aspect of the invention there is provided a catheter assembly comprising: a catheter comprising a proximal end for insertion into the body and a distal end, a sleeve configured to enclose the catheter, and a wetting mechanism arranged at the proximal end of the catheter, wherein the wetting mechanism comprises a base and a body, the body comprises a fluid reservoir, the base and body are rotatable with respect to one another to define a first configuration and a second configuration of the wetting mechanism, wherein in the second configuration the wetting mechanism permits release of fluid from the fluid reservoir into the sleeve to wet the catheter.

Advantageously, as the wetting mechanism is placed at the proximal end of the catheter the first part of the catheter to enter the body is the part most likely to be wetted by the wetting mechanism. In addition, the wetting mechanism is easier and more intuitive to use as the user can simply rotate the base and body with respect to one another to release wetting fluid from the fluid reservoir. This is an easier and simpler task that can be done in a more controlled manner even for users with reduced manual dexterity. This increases the likelihood that the user will use the wetting mechanism as intended rather than attempting to access the catheter by other means which may not result in the catheter being adequately wetted and kept clean prior to use. This helps reduce the likelihood of discomfort, injury and infection during use.

In the first configuration the wetting mechanism may inhibit release of fluid from the fluid reservoir to wet the catheter. In the first configuration the wetting mechanism may inhibit release of fluid from the fluid reservoir into the sleeve to wet the catheter. Thus, the release of wetting fluid is controlled to ensure that the catheter is not prematurely wetted.

The wetting mechanism may be tubular. An axial direction may be defined by along the axis of the wetting mechanism. The body and base may rotate with respect to one another in a plane perpendicular to the axial direction. The wetting mechanism may have an outer peripheral shape in a plane perpendicular to the axis of the wetting mechanism (e.g. the axial direction). The outer peripheral shape may have rotational symmetry of order 2 or more. The outer peripheral shape may have rotational symmetry of less than order infinity, or less than order 10, or less than order 8 or less than order 6. The wetting mechanism may have the same outer peripheral shape in the first and second configurations. The wetting mechanism may have a cross-section defining the outer peripheral shape of the wetting mechanism. Cross-section as mentioned herein generally refers to the outer peripheral shape of an object in a plane perpendicular to the axial direction unless mentioned otherwise. The rotational symmetry of the outer peripheral shape/cross-section is preferably of order 2. The wetting mechanism may have any suitable shape or size cross-section to define the outer peripheral shape, for example, elliptical, rectangular, square or irregularly shaped, preferably the cross-section is elliptical. The outer peripheral shape may have a major axis and a minor axis A major axis may be defined by the widest point of the outer peripheral shape. A minor axis may be defined by the narrowest point of the outer peripheral shape. The major and minor axis may preferably be orthogonal but may be arranged at an acute angle with respect to each other. A major axis and minor axis may be defined by the elliptical cross-section of the wetting mechanism. The major axis may comprise a vertex at each end. The minor axis may comprise a co-vertex at each end. The major axis may be no more than 2, 3, 4 or 5 cm. The major axis may be no less than 1, 2, 3, or 4 cm. Preferably, the major axis is 2-4 cm, for example 3.5 cm. The minor axis is less than the major axis, for example, 5%, 10%, 15% or 20% less than the major axis or 1.5-3.5 cm for example 3.1 cm. Preferably, the minor axis is 10% less than the major axis. Thus, the wetting mechanism may have a comfortable shape that is easy for the user to hold and it is easy for the user to identify if the wetting mechanism is in the first or second configurations due to the rotational symmetry of the wetting mechanism.

The body may be tubular. The body may comprise a cross-section that is the same as the cross-section of the wetting mechanism. The body may have a major axis that corresponds to the major axis of the wetting mechanism. The body may have a minor axis that corresponds to the minor axis of the wetting mechanism. The body may have a length that is larger than its major axis, for example 3-10% larger and preferably 5% larger, for example 4 cm. The body may comprise an outer tubular shell that forms the tubular shape of the body. The outer tubular shell may have a thickness of 3-5% of the major axis, for example 4% or 0.5 mm.

The base may be tubular. The base may comprise a cross-section that is the same as the cross-section of the wetting mechanism. The base may have a major axis that corresponds to the major axis of the wetting mechanism. The base may have a minor axis that corresponds to the minor axis of the wetting mechanism. The base may have a length that is smaller than its major axis. The body may be longer than the base. The base may have a length that is smaller than the length of the body, for example 40-50% or 45% of the length of the body, for example 2 cm. The body base comprise an outer tubular shell that forms the tubular shape of the base. The outer tubular shell may have a thickness of 3-5% of the major axis, for example 4% or 0.5 mm.

The length of the body and base may be measured in an axial direction that is parallel to the axis of rotation of the body with respect to the base. The length of the body and base may be measured along a direction in which the catheter passes through the wetting mechanism. The length of the body and base may be measured along a direction parallel to the catheter when inside the wetting mechanism. The length of the body/base may include all elements of the body/base respectively, for example all parts integrally formed with the body/base.

The body may define part of the outer peripheral shape of the wetting mechanism. The body may define substantially all the outer peripheral shape of the wetting mechanism along a section of the length of the wetting mechanism. The base may define part of the outer peripheral shape of the wetting mechanism. The base may define substantially all the outer peripheral shape of the wetting mechanism along a section of the length of the wetting mechanism. The body and base may define different, and preferably adjacent, parts of the outer peripheral shape of the wetting mechanism. The base may define a part of the outer peripheral shape of the wetting mechanism between the body and the sleeve. When in the first configuration, the outer peripheral shape of the body may match that of the base. When in the second configuration, the outer peripheral shape of the body may match that of the base. The outer peripheral shape of the body may not match that of the base when the body/base are at a point of rotation between the first and second configurations, for example in the third configuration as described below. The outer peripheral shape of the body and base may match when they are, for example, aligned, continuous, smooth, and/or flush at an interface between the body and base.

The wetting mechanism may be symmetric about the major axis. In the first configuration the major axis of the base and the major axis of the body may be aligned. In the second configuration the major axis of the base and the major axis of the body may be aligned, preferably, with one of the base or body pointing the opposite direct to the first configuration. When not in the first or second configuration, the major axis of the base may not be aligned with the major axis of the body. The major axis of the base may not be aligned with the major axis of the body at a point of rotation between the first and second configurations. The major axis of the base may be aligned with the minor axis of the body at a point of rotation between the first and second configurations. Thus, the user can easily identify if the wetting mechanism is in the first or second configurations or neither of them.

The body may comprise a body aperture. The body aperture may be configured to allow the catheter to pass into and/or out of the body. The body aperture may be a body guide tube. The body guide tube may extend in the axial direction through the body. The body aperture/body guide tube may be configured to direct the catheter through the body. The guide tube may be open-ended. The guide tube may be cylindrical. The guide tube may have a diameter that is 20-40% of the major axis, for example, 30% or 1 cm. The guide tube may be located between the centre of the body and a vertex of the major axis, preferably the guide tube is located midway between the centre and a vertex. Thus, the catheter may be easily and safely passed through the body.

The body may comprise a divider. The divider may extend in the axial direction through the body. The divider may define the fluid reservoir. The divider may be configured to separate the internal volume of the body. The divider may be configured to separate the guide tube from the fluid reservoir. The divider may extend around the guide tube, for example in a plane perpendicular to the axial direction. The divider may have an arched cross-section. The divider may extend from the outer tubular shell of the body, preferably either side of the guide tube. The divider may extend to a point 55-65%, or 57. %-62.5% of the way along the major axis of the body from the vertex (or an edge where the wetting mechanism is not elliptical) adjacent the guide tube, for example 60% or 2 cm. A tip of the divider may be defined at the point it crosses the major axis. The curvature of the divider may be a maximum at the tip. The divider may extend tangentially from either side of the guide tube. The curvature of the divider may be a minimum where it meets the outer tubular shell of the body. The divider and guide tube may be independent. The divider and guide tube may be integrally formed. Thus, the fluid reservoir and guide tube are divided within the body.

The body may comprise a body wall. The body wall may cap one end of the body. The other end of the body may be open ended, that is uncapped. The body aperture may be located in the body wall. The body guide tube may extend from the body wall. The divider may extend from the body wall. The body guide tube may extend less than 100%, 98%, 95%, or 90% of the length of the body. The divider may extend less than 100%, 98%, 95% or 90% of the length of the body. The body guide tube and divider may be substantially the same length.

The body may comprise an axle. The axle may be configured to allow the body and base to rotate with respect to each other, preferably about an axis defined by the axial direction. The axle may engage the base. The axle may extend parallel to the axial direction. The axle may be arranged at the centre of the cross-section of the body. The axle may be arranged at the centre of the body wall. The axle may be cylindrical. The axle may have a diameter of 15-25% of the major axis of the body, for example 20% or 7 mm. The axle may have open ends. The axle may have a length of 20-40% of the body, for example 30%.

The axle may comprise at least two slits, for example four slits. The at least two slits may be arranged with equal separations around the circumference of the axle, for example a 90degree separation where there are four slits. Each slit may extend in an axial direction from an end of the axle distal to the body. Each slit may extend 35-40% of the length of the axle, for example 37.5%. A locking protrusion may be present between two adjacent slits. The axle may comprise at least one locking protrusion. Each locking protrusion may be configured to engage the base. Preferably, a locking protrusion is present between every pair of adjacent slits. Preferably, there are the same number of slits and locking protrusions. Each locking protrusion may be arranged at the end of the axle distal to the body. Each locking protrusion may span 15-25% of the length of the axle for example 20% or about half the length of the slit. Each protrusion may be narrowest at the end of the axle distal to the body. Each protrusion may be wedge shaped. The effective diameter of the axle may increase linearly by 35-45%, for example 40%, due to the one or more locking protrusions. Thus, the slits allow the locking protrusions to move and engage the base as described below.

The base may comprise a base aperture configured to allow the catheter to pass in to, through, and/or out of the base. The base aperture may be sized to allow passage of the catheter therethrough. The base aperture may be provided by a base guide tube. The base guide tube may extend in the axial direction through the base. The base guide tube may be configured to direct the catheter through the base. The base guide tube may be open-ended. The base guide tube may be cylindrical. The base guide tube may have a diameter that is substantially the same as the body aperture/body guide tube, that is 20-40% of the major axis, for example, 30% or 1 cm. The base guide tube may be located between the centre of the base and a vertex of the major axis, preferably the base guide tube is located midway between the centre and a vertex. Thus, the catheter may be easily and safely passed through the base.

The base may comprise a base wall. The base wall may cap one end of the base. The other end of the base may be open ended, that is uncapped. The base guide tube may extend from the base wall. The base guide tube may extend over the entire length of the base from the base wall.

The base may be configured to receive the axle. The base may comprise a locking aperture configured to receive the axle. The locking aperture may be provided on the base wall. The locking aperture may be provided centrally on the base wall. The locking aperture may be tubular, for example cylindrical. The locking aperture may extend in the axial direction. The locking aperture may extend within the body. The locking aperture may extend away from the base wall. The locking aperture may have a length in the axial direction equivalent to the distance between the body wall the locking protrusions of the axle. Thus, the body and base may be attached together using the axle and locking aperture.

As described above, the axle and locking aperture work together to secure the base and body together. While the axle is described as being part of the body and the locking aperture part of the base, there is no reason why this relationship could be reversed and the axle be a part of the base and extend from the base wall and the locking aperture be provided in the body, such as in the body wall.

The fluid reservoir may comprise an opening to allow wetting fluid to be released from the fluid reservoir. The opening may be provided in the body. The opening may be provided in the body wall. The opening may be an outlet. The outlet may be circular. The outlet may have a diameter of 60-70%, for example 65%, of the diameter of the body aperture/body guide tube. The opening may be positioned midway between a co-vertex and the centre of the body. The opening may have an angular separation around the cross-section of the body of 80-100 degrees, for example 90 degrees, from the body aperture/body guide tube.

The fluid reservoir may comprise a sealing element. The sealing element may be configured to seal the opening. The sealing element may be configured to provide a seal between the opening and the base, for example when the wetting mechanism is in the first configuration. The sealing element may be configured to inhibit passage of fluids between the base and body. The sealing element may be formed of a flexible material such as a flexible plastics material, rubber or silicone. The sealing element may be any suitable shape or size to inhibit flow of fluid out of the fluid reservoir until the wetting mechanism is in the second configuration. The sealing element may be an O-ring. Where the sealing element is an O-ring, the sealing channel may be annular. Thus, the sealing element is simple and easy to manufacture while providing an effective seal.

The sealing element may be compressed between the base and the body. The sealing element may be compressed in the axial direction. The axle may urge the body into the base so as to compress the sealing element. Each locking protrusion may urge the sealing element into compression between the body and base. The sealing element may be compressed by at least 1%, 5%, 10%, 20%, or 30% in the axial direction. The sealing element may be compressed by no more than 40%, 30%, 20%, 10%, 5%, or 1% in the axial direction.

The sealing element may be configured to provide a frictional force to inhibit rotation of the base with respect to the body. The sealing element may provide a frictional force between the base and the body equivalent to at least 0.05 Nm, 0.1 Nm, 0.2 Nm, 0.3 Nm, 0.4 Nm, 0.5 Nm or 1 Nm of torque about the centre of rotation of the base with respect to the body, that is the centre of the wetting mechanism. The sealing element may provide a frictional force between the base and the body equivalent to no more than 1 Nm, 0.5 Nm, 0.4 Nm, 0.3 Nm, 0.2 Nm, 0.1 Nm, or 0.05 Nm of torque about the centre of rotation of the base with respect to the body, that is the centre of the wetting mechanism. Preferably, the sealing element provides a frictional force between the base and the body equivalent to no more than 0.35 Nm of torque about the centre of rotation of the base with respect to the body, that is the centre of the wetting mechanism. Thus, the wetting mechanism is not inadvertently changed configuration which could cause premature wetting of the catheter.

The fluid reservoir may comprise a retainer configured to restrict movement of the sealing element with respect to the opening. The retainer may be provided in the body. The retainer may be provided in the body wall. The retainer may be configured to urge the sealing element into a compressive seal between the base and body. The retainer may be a sealing channel. The sealing channel may be arranged around the opening, preferably concentrically around the opening. The sealing channel may overlap with the outer tubular shell of the body. The outer tubular shell may thin to accommodate the sealing channel. The retainer may be independent from the opening. The sealing channel may be configured to receive the sealing element. The sealing channel may have a depth that is less than a thickness of the sealing element. Thus, a secure seal is provided through the retainer.

The base may comprise an outlet opening. The outlet opening may be configured to provide a fluid connection between the base and the fluid reservoir. The outlet opening may provide a fluid connection between the base and the fluid reservoir when the wetting mechanism is in the second configuration. The outlet opening may be configured to direct wetting fluid onto the catheter to wet the catheter, preferably when the wetting mechanism is in the third and/or second configurations. Most preferably, the outlet opening is configured to direct wetting fluid onto the catheter to wet the catheter when the wetting mechanism is in the third or second configurations. This helps to ensure the catheter is wetted before it can be inserted into the body and that wetting fluid can continue to flow onto the catheter while the catheter is being used.

The outlet opening may not be aligned with the opening when the wetting mechanism is in the first configuration. The outlet opening may be aligned with the opening when the wetting mechanism is in the second configuration. The outlet opening may comprise an arced opening in the base wall. The outlet opening may extend through an arc equivalent to an angle of up to 160, 150, 140, 130, 120, 110, or 100 degrees around the centre of the base, for example 150-160 degrees around the axis of rotation of the body and base with respect to each other, for example around the centre of base. The outlet opening may start 40 degrees to one side of the base guide tube as measured from the centre of the base guide tube. The outlet opening may terminate 110 degrees to the other side as measured from the centre of the base guide tube.

The outlet opening may span a radius that corresponds to the position of the opening. The outlet opening may span from a radius of 40% of the minor axis of the base to a radius of 60% of the minor axis of base. The outlet opening is thereby aligned with the outlet when the body rotates with respect to the base as described below.

The outlet opening may overlap with the base aperture/base guide tube. The outlet opening may be a different size/shape to the base aperture/base guide tube. The outlet opening may be a different size/shape to the opening. The outlet opening may be at least partially non-overlapping with the base aperture/base guide tube. The base aperture/base guide tube may intersect the outlet opening. The base aperture/base guide tube may split the outlet opening into two or more portions. The base aperture/base guide tube may split the outlet opening into three portions. One of the portions may be the base aperture/base guide tube itself. One of the portions may be on one side of the base aperture/base guide tube. Another of the portions may be on an opposite side of the base aperture/base guide tube. The base aperture/base guide tube and outlet opening may not provide a continuous volume within the base. Thus, the base guide tube remains a cylindrical tube and is not affected by the outlet opening's shape which ensures the catheter can effectively travel through the base guide tube.

As described above, the opening, outlet opening and retainer work together to selectively allow draining of the fluid reservoir into the base. While the opening and retainer are described as being part of the body and the outlet opening part of the base, there is no reason why this relationship could be altered. For example, a retainer could be provided on the body, the base or both the body and the base. In addition, the relative sizes and shapes of the opening and outlet opening could be reversed and the same or equivalent functionality obtained. In addition, the sealing element could comprise multiple sealing elements, for example one retained on the body and one on the base which work together to provide the required fluid-tight sealing in the first configuration.

The wetting mechanism may comprise a rotation guide configured to restrict linear movement of the base with respect to the body during rotation of the base with respect to the body. The rotation guide may comprise at least two interlocking members. The body may comprise one interlocking member. The base may comprise one interlocking member. The at least two interlocking members may comprise a pin and a slot. The body may comprise the pin. The body wall may comprise the pin. The pin may extend in the axial direction from the body wall. The pin may extend away from the body. The pin may be cylindrical. The pin may have capped ends. The pin may have a diameter of 5-10%, for example 7.5%, of the major axis of the body or 5 mm. The pin may have a length of 30-40%, for example 35%, of the length of the axle. The pin may have an angular separation around the cross-section of the body of 110-150 degrees, for example 130 degrees, from the body aperture/body guide tube. The pin may have an angular separation around the cross-section of the body of 120-160degrees, for example 140 degrees, from the opening. The pin may be positioned approximately 30-50%, for example 40%, of the distance from the centre of the axle to the outer tubular shell of the body as measured through the pin.

The base may comprise the slot. The slot may be configured to receive the pin. The slot may be provided in the base wall. The slot may have a depth that is equivalent to the length of the pin. The slot may be configured to allow the pin can travel along slot as the base and body are rotated with respect to one another. The slot may be arced. The slot may span a radius that corresponds to the diameter of the pin. The slot may span from a radius of 30% to a radius of 55% of the minor axis of the base. The slot may cover an arc length of at least 180degrees around the base, for example 210-220 degrees. The slot may cover an arc length of 180 degrees plus the angular size of the pin. The slot may start on one side of the base guide tube. The slot may overlap with the outlet opening. The slot and outlet opening may define a continuous volume. Thus, the pin and slot may allow the body and base to rotate with respect to one another by up to 180 degrees.

The rotation guide may be configured to provide a force to inhibit rotation of the base with respect to the body, preferably a frictional force. The rotation guide and sealing element may be configured to each independently provide a force to inhibit rotation of the base with respect to the body. The rotation guide may be configured to provide a force to inhibit rotation of the base with respect to the body only over part of the range of rotation of the body with respect to the base, and preferably over a minority of the range of rotation. The rotation guide may provide a maximum force to inhibit rotation of the base with respect to the body as the mechanism enters and/or leaves the second configuration. The rotation guide may provide a maximum force to inhibit rotation of the base with respect to the body as the mechanism leaves the second configuration. The rotation guide may provide a force between the base and the body equivalent to at least 0.05 Nm, 0.1 Nm, 0.2 Nm, 0.3 Nm, 0.4 Nm, 0.5 Nm or 1 Nm of torque about the centre of rotation of the base with respect to the body, that is the centre of the wetting mechanism. The rotation guide may provide a force between the base and the body equivalent to no more than 1 Nm, 0.5 Nm, 0.4 Nm, 0.3 Nm, 0.2 Nm, 0.1 Nm, or 0.05 Nm of torque about the centre of rotation of the base with respect to the body, that is the centre of the wetting mechanism. Thus, the wetting mechanism is not inadvertently entered into, or removed from, the second configuration which could cause damage to the catheter. In addition, the user is able to tell when the wetting mechanism is about to enter the second configuration as they must overcome the additional force provided by the rotation guide to enter the second configuration.

The wetting mechanism may be configured to provide audible/tactile feedback as the wetting mechanism enters and/or leaves the second configuration. The rotation guide may be configured to provide audible/tactile feedback as the wetting mechanism enters and/or leaves the second configuration. The at least two interlocking members may be configured to provide audible/tactile feedback as the wetting mechanism enters and/or leaves the second configuration. The slot may comprise one or more slot protrusions. The slot protrusions may be configured to inhibit movement of the pin along the slot and past the slot protrusions. The slot protrusions may be configured to exert a force on the pin to inhibit rotation of the body with respect to the base. The slot protrusions may provide audible/tactile feedback when the pin passes the slot protrusions, for example due to deformation of the slot protrusions, pin, body and/or base. The slot protrusions may extend into the slot in a direction perpendicular to the axial direction. The slot protrusions may extend in a plane parallel to the plane of rotation of the body with respect to the base. The slot protrusions may be positioned towards an end of the slot distal from the base guide tube. The slot protrusions may be separated from an end of the slot by a distance equivalent to the diameter of the pin, for example an angular distance of 30-40 degrees. Thus, the user is provided with feedback to let them know the wetting mechanism is in the second configuration and also to ensure that the wetting mechanism neither inadvertently enters the second configuration nor leaves it. In addition, as the slot protrusions extend into the slot in a direction perpendicular to the axial direction, then the slot protrusions do not require the pin to move axially to overcome them. This avoids the slot protrusions causing unintentional axial forces whilst they are overcome which could cause separation of the base and body and damage to the wetting mechanism.

As described above, the pin and slot work together to guide rotation of the base and body and provide audible/tactile feedback. While the pin is described as being part of the body and the slot part of the base, there is no reason why this relationship could be reversed and the pin be a part of the base and extend from the base wall and the slot be provided in the body, such as in the body wall. In addition, other aspects of the wetting mechanism could be adapted to provide audible/tactile feedback such as through dedicated detents/protrusions.

In the first configuration, the pin may be arranged at an end of the slot adjacent to the base guide tube. In the first configuration the wetting mechanism may prevent passage of the proximal end of the catheter therethrough. In the first configuration, the body aperture/body guide tube may not be aligned with the base aperture/base guide tube. In the first configuration, the opening may be sealed by the sealing element, for example, the sealing element may seal between the outlet and the base wall.

In the first configuration, the body aperture/body guide tube may be a misaligned with the base aperture/base guide tube to prevent passage of the proximal end of the catheter therethrough. Thus, the movement of the catheter through the wetting mechanism is blocked as the apertures/tubes are misaligned.

In the second configuration, the pin may be arranged at an end of the slot distal from the base guide tube. In the second configuration the wetting mechanism may permit passage of the proximal end of the catheter therethrough. In the second configuration, the pin may be retained in position by the slot protrusions. In the second configuration, the body aperture/body guide tube may be aligned with the base aperture/base guide tube to permit passage of the proximal end of the catheter therethrough. In the second configuration, the opening may be aligned with an end of the outlet opening.

Accordingly, in a preferred embodiment, there is provided a catheter assembly comprising: a catheter comprising a proximal end for insertion into the body and a distal end; and a housing, wherein the housing comprises a base and a body, the base and body are rotatable with respect to one another to define a first configuration and a second configuration of the housing, wherein in the first configuration the housing prevents passage of the proximal end of the catheter therethrough and in the second configuration the housing permits passage of the proximal end of the catheter therethrough wherein the base comprises a base aperture sized to allow passage of the catheter therethrough and wherein the body comprises a body aperture sized to allow passage of the catheter therethrough, wherein in the first configuration the body aperture is misaligned with the base aperture to prevent passage of the proximal end of the catheter therethrough and in the second configuration the body aperture is aligned with the base aperture to permit passage of the proximal end of the catheter therethrough. Thus, the housing is able to simply and effectively permit or prevent passage of the proximal end of the catheter therethrough.

In another preferred embodiment, there is provided a catheter assembly comprising: a catheter comprising a proximal end for insertion into the body and a distal end; and a housing, wherein the housing comprises a base and a body, the base and body are rotatable with respect to one another to define a first configuration and a second configuration of the housing, wherein in the first configuration the housing prevents passage of the proximal end of the catheter therethrough and in the second configuration the housing permits passage of the proximal end of the catheter therethrough wherein the base comprises a base guide tube sized to allow passage of the catheter therethrough and wherein the body comprises a body guide tube sized to allow passage of the catheter therethrough, wherein in the first configuration the body guide tube is misaligned with the base guide tube to prevent passage of the proximal end of the catheter therethrough and in the second configuration the body guide tube is aligned with the base guide tube to permit passage of the proximal end of the catheter therethrough. Thus, the housing is able to simply and effectively permit or prevent passage of the proximal end of the catheter therethrough and further the provision of guide tubes ensures that the proximal end of the catheter passes smoothly through the housing with a lower risk of the catheter catching or getting stuck.

The wetting mechanism may comprise a third configuration between the first and second configurations. In the third configuration, the major axes of the body and base may not be aligned. In the third configuration, the outer peripheral shape defined by the body and base may not match. In the third configuration the fluid reservoir may be configured to release fluid into the sleeve to wet the catheter. In the third configuration the wetting mechanism may prevent passage of the proximal end of the catheter therethrough. In the third configuration, the pin may be arranged between either end of the slot. In the third configuration, the opening/outlet may be aligned with at least part of the outlet opening. In the third configuration, the body aperture/body guide tube may not be aligned with the base aperture/base guide tube.

Thus, in a preferred embodiment, there is provided a catheter assembly comprising: a catheter comprising a proximal end for insertion into the body and a distal end; and a housing, wherein the housing comprises a base and a body, the base and body are rotatable with respect to one another to define a first configuration and a second configuration of the housing, wherein in the first configuration the housing prevents passage of the proximal end of the catheter therethrough and in the second configuration the housing permits passage of the proximal end of the catheter therethrough wherein the body comprises a fluid reservoir and the housing comprises a third configuration between the first and second configurations wherein, in the first configuration the housing inhibits release of fluid from the fluid reservoir to wet the catheter, in the third configuration the fluid reservoir is configured to release fluid into the sleeve to wet the catheter and prevent passage of the proximal end of the catheter therethrough. Advantageously, this ensures wetting fluid is released to wet the catheter before the catheter can be moved through the housing. This improves the likelihood that it is properly lubricated before use.

The wetting mechanism may comprise an adapter. The adapter may be configured to provide a fluid connection between the wetting mechanism and sleeve. The adapter may be configured to be attached to the base. The adapter may be configured to be attached to the base distal from the body. The adapter may be configured to be attached to the base distal from the base wall. The sleeve may be attached to the adapter, for example by any suitable means such as a weld; mechanical seal; heat seal; pressure seal; adhesive; solvent bond; ultraviolet bond; ultrasonic weld; laser weld; impulse weld; or friction weld. Thus, the adapter ensures the sleeve is securely attached to the wetting mechanism.

The base may be provided between the body and the sleeve. The sleeve may be attached to the base, for example via the adapter. The body may be attached to the base, for example as described above. The body may be attached to the sleeve via the base. The base may be fixed with respect to the sleeve. Of course, the sleeve is typically flexible and so parts of the sleeve may be able to move with respect to the base, however, where the base is fixed with respect to the sleeve, it is at least fixed relative to an the end of the sleeve. The body may rotate on the base.

The adapter may be configured to guide the catheter into the wetting mechanism, for example into the base guide tube. This helps the catheter to pass through the wetting mechanism and improves the efficiency of the catheter wetting process.

The adapter may comprise an adapter wall. The adapter wall may have an outer edge that corresponds to the cross-section of the outer tubular shell of the body and/or the cross-section of the wetting mechanism. The adapter may therefore have a major axis that corresponds to the major axis of the wetting mechanism. The adapter may have a minor axis that corresponds to the minor axis of the wetting mechanism. Thus, the adapter provides a smooth outer appearance and feel to the wetting mechanism in conjunction with the other parts of the wetting mechanism.

The adapter may direct wetting fluid onto the catheter and/or into the sleeve. The adapter wall may direct wetting fluid onto the catheter and/or into the sleeve. The adapter wall may comprise a flat portion. The adapter wall may comprise a recessed portion. The recessed portion may be shaped to direct wetting fluid into the sleeve and/or onto the catheter. The recessed portion may extend away from the base. The flat portion may be perpendicular to the axial direction. An outer edge of the adapter wall may be in the plane defined by the flat portion. The flat portion and recessed portion may be separated along a line approximately joining the co-vertices of the adapter. The line may be smoothly arced. The flat portion may encompass one vertex and the recessed portion may encompass the other vertex. The flat portion may encompass both co-vertices. The recessed portion may encompass the centre of the adapter.

The adapter may comprise an adapter tube. The adapter tube may be attached to the recessed portion. The adapter tube may provide access for the catheter through the adapter, optionally via the recessed portion. The adapter tube may provide access for wetting fluids through the adapter, optionally via the recessed portion. The adapter tube may be cylindrical. The adapter tube may be open-ended. The adapter tube may have a diameter larger than the base guide tube diameter. The adapter tube may have a diameter of 20-30% the major axis, for example 25%. The adapter tube may have a length of 45-55%, for example 50% of the major axis of the adapter. The adapter tube may extend from the recessed portion. The adapter tube may extend in the axial direction. The adapter tube may extend away from the flat portion. The adapter tube may be aligned with the base guide tube. The adapter tube may be positioned between the vertex encompassed by the recessed portion and the centre of the adapter. The adapter tube may be positioned 70-90%, for example 80%, of the way between the two vertices of the adapter. Thus, the adapter tube ensures smooth and efficient transfer of the catheter and wetting fluids between the sleeve and wetting mechanism.

The recessed portion may be is recessed by a maximum distance of 20-30%, for example 25% of the major axis of the adapter from the flat portion. An end of the adapter tube distal from the flat portion may be a distance from the flat portion equivalent to 65-85%, for example 75% of the major axis of the adapter. The recessed portion may have a continuous smooth surface. The recessed portion may extend smoothly between the adapter tube and the flat portion. The adapter tube may be arranged at the point on the recessed portion where it is a maximum distance from the flat portion. The recessed portion may be curved, for example due to the arced interface between the recessed and flat portions. Advantageously, the curved shape assists in directing wetting fluid into the adapter tube and on into the sleeve and/or onto the catheter.

The adapter may comprise one or more sealing ribs. The one or more sealing ribs may be configured to provide a fluid-tight seal between the adapter and the base. Each sealing rib may be arranged on the inside of the outer edge of the adapter wall. Each sealing rib may extend in the axial direction. Each sealing rib may extend away from the adapter tube. Each sealing rib may terminate at the same distance from the flat portion as measured in the axial direction, for example a distance of 10% the major axis of the adapter. Each sealing rib may be chamfered. Each sealing rib may engage the base. Each sealing rib may extend around an arc length about the centre of the adapter. Each sealing rib may extend between 10-360 degrees around the adapter. Preferably, the one or more sealing ribs comprise two minor sealing ribs. Each minor sealing rib may extend around an arc length of 30 degrees. The minor sealing ribs may be provided on either side of the adapter tube. The minor sealing ribs may be separated by an angle of 60 degrees. The one or more sealing ribs may comprise a major sealing rib. The major sealing rib may extend around an arc length of 160 degrees. The major sealing rib may be centred on the vertex encompassed by the flat portion. The minor sealing ribs may each be separated from an edge of the major sealing rib by 40 degrees. Thus, the sealing ribs may be used to efficiently seal the adapter to the base by pushing them together.

The wetting mechanism may comprise an inserter tip. The inserter tip may therefore have a major axis that corresponds to the major axis of the wetting mechanism. The inserter tip may have a minor axis that corresponds to the minor axis of the wetting mechanism. The inserter tip may be configured to seal an end of the body distal from the base. The inserter tip may be configured to allow the catheter to exit the wetting mechanism and pass into the body. The inserter tip may comprise an insertion tube. The insertion tube may be cylindrical. The insertion tube may have an inner diameter that is larger than the outer diameter of the catheter, for example 20-30% of the major axis, for example 25% or 7 mm. The insertion tube may have a length that is 50-60% of the major axis, for example 55%. The insertion tube may have a constant thickness that is also the same as the wall thickness of the other parts of the inserter tip, and optionally the rest of the wetting mechanism, of 1 mm. The insertion tube may be arranged parallel to the axial direction. The insertion tube may be aligned with the body aperture/body guide tube. The insertion tube may be configured to be inserted into the urethra during use such that the catheter passes directly from the wetting mechanism into the user's body. Thus the inserter tip helps to reduce the risk of infection and discomfort as the catheter is guided smoothly into the body by the insertion tube.

The insertion tube may be capped at one end. The insertion tube may be capped by a hemicylindrical dome. The hemicylindrical dome may comprise at least one slit, for example two orthogonal slits. The at least one slit may be arranged centrally on the dome. The at least one slit may define at least one flap, for example four flaps. The at least one slit may be configured to allow the at least one flap to separate. The at least one flap may separate to allow the proximal end of the catheter to exit the wetting mechanism through the insertion tube. Thus, the domed shape helps to facilitate comfortable insertion of the insertion tube into the body if required and the catheter may be easily passed into the body via the slits/flaps.

The inserter tip may comprise a tip wall. The tip wall may be configured to cap the end of the body distal from the body wall. The tip wall may have a thickness of 5% of the length of the insertion tube. The insertion tube may extend out of a proximal surface of the tip wall distal from the body. The insertion tube may not extend from the tip wall in the opposite direction, for example from an opposite distal surface of the tip wall. Thus, the tip wall helps to inhibit over insertion of the insertion tube into the body while also sealing the inserter tip to the body.

The inserter tip may comprise a seat. The seat may be configured to be received by the body. The seat may abut the body guide tube and/or divider. The seat may extend from the tip wall and preferably from the distal surface of the tip wall. The seat may surround the insertion tube on the tip wall. The seat may provide a region of the tip wall that is 60-100% thicker than the rest, for example 80%. The seat may direct the catheter into the insertion tube. The seat may have a chamfered edge, for example where it surrounds the insertion tube. Thus the seat assists with passage of the proximal end of the catheter into the insertion tube and helps securely seal the inserter tip to the body.

The seat may have a shape that matches the shape of the divider and/or guide tube. The seat may have the general shape of an isosceles triangle. The seat may have convex sides. The seat may have rounded corners. A gap may be provided between the seat 2126 and an outer edge of the tip wall to accommodate the outer tubular shell of the body, for example the gap may be approximately 3-5% of the major axis of the tip wall 2125, for example 4%. The seat may extend into and past the centre of the tip wall. The seat may have a height in a direction parallel to the major axis of 55-60% of the major axis of the tip wall 2125, for example 57%. The seat may have a width perpendicular to its height that is at a maximum approximately inline with the centre of the insertion tube along the major axis of the tip wall. The seat width may be 70-80% of the seat 2126 height, for example 75%. Thus, the seat matches the shape of the divider and ensures a tight seal therebetween.

The inserter tip may comprise a sealing rib. The sealing rib may extend from the distal surface of the tip wall. The sealing rib may form a continuous loop. The sealing rib may follow the shape of the seat and/or divider into the centre of the tip wall. The sealing rib may extend around the perimeter of the tip wall that is not occupied by the seat. The gap between the seat and the edge of the tip wall may be maintained between the sealing rib and the edge of the tip wall. The sealing rib may extend from the distal surface a distance of 2-3 times the thickness of the tip wall 2125, for example 2.5 times. The sealing rib may have a chamfered edge distal from the distal surface. Thus, the sealing rib may engage the outer tubular shell of the body along with the divider to provide a tight seal and define part of the fluid reservoir.

The inserter tip may comprise a detent. The detent may be configured to engage the cap as described below. The detent may be a through-hole that extends completely through the tip wall. The detent may also extend part of the way through the tip wall. The detent may be positioned approximately midway between a vertex and the centre of the inserter tip. The detent may be positioned on an opposite side of the inserter tip from the insertion tube. The detent may be circular with a diameter of approximately half to one third the diameter of the insertion tube.

The wetting mechanism may comprise a cap. The cap may be configured to cover the inserter tip. The cap may be configured to protect the inserter tip before use. The cap may comprise a shell with the same shape as the inserter tip. The cap may be larger than the inserter tip such that it can enclose it.

The cap may comprise a cap tube. The cap tube may be configured to overlie the insertion tube. The cap tube may be cylindrical. The cap tube may comprise an inner diameter that matches, or is larger than, the outer diameter of the insertion tube. The cap tube may be capped at one end, for example with a hemicylindrical dome to fit over the dome of the insertion tube.

The cap may comprise a cover. The cover may extend out from the open end of the cap tube. The cover may be configured to overlie the tip wall. The cover may have an outer perimeter that is just outside the outer perimeter of the inserter tip and/or the body, for example, the major axis of the cap may be 10% larger than the major axis of the inserter tip and body.

The cap may comprise a cap flange. The cap flange may extend from the outer edge of the cover. The cap flange may extend in the axial direction to overlie the inserter tip and optionally the body. The cap flange may have a length such that when the cap is placed over the inserter tip, the cap flange extends down past the distal surface of the tip wall approximately 50-70% of the distance the sealing rib extends from the distal surface, for example 60%.

The free end of the cap flange may comprise a gripping protrusion. The gripping protrusion may extend around the outer perimeter of the cap flange. The gripping protrusion may occupy the bottom 20-30% of the cap flange, for example the bottom 25% and has a semi-circular cross-section. In the region of the gripping protrusion, the cap flange may have a major axis that is 3% larger than the rest of the cap flange. The gripping protrusion thereby helps ensure the cap remains attached to the wetting mechanism and also assists the user in removing the cap when required.

The cap may comprise a plug. The plug may extend from the cover. The plug may be configured to be received by the detent. Where the detent is a through-hole, the plug may be configured to seal the through-hole. The plug may be shaped to be slightly larger than the detent where it meets the cover. The plug may have a frustoconical shape. The plug may bear against the inside of the detent once received within it. Thus the through-hole may be sealed to inhibit leakage of wetting fluid out of the fluid reservoir. In addition, removal of the plug from the through-hole allows the user to provide an air intake for the fluid reservoir that assists with release of wetting fluid into the sleeve as air can pass into the fluid reservoir to replace the wetting fluid that is leaving. The plug also helps to retain the cap on the inserter tip.

The cap may comprise a pull-ring. The pull-ring may be configured to allow the user to grasp the cap and pull it off the wetting mechanism. The pull-ring may be attached to the dome, cap tube, cover or any suitable part of the cap. Where the pull-ring is attached to the dome and/or cap tube, the pull-ring may be arranged off-centre with respect to the cap tube. The pull-ring may be centrally located with respect to the centre of the cap. This helps the user to remove the cap as the position of the pull-ring reduces shearing forces that are not parallel to the axial direction which can cause the cap to remain stuck on the wetting mechanism. Of course, in other embodiments, a different easily gripped feature may be used in place of the pull ring, such as a tab and the pull-ring or equivalent feature may be placed in a different position such as directly attached to the cover or cap flange.

The pull-ring may comprise a reinforced region. The reinforced region may encompass half of the pull-ring, for example the half distal from the cap. The reinforced region may comprise a strengthened region of the pull-ring. The reinforced region may therefore allow a greater force to be exerted onto the cap by the user. The reinforced region may have a widened cross-section compared to the rest of the pull-ring. The reinforced region may have a square cross section.

The cap, inserter tip, body, base and/or adapter may each comprise a material that is more rigid than the sealing element, for example a hard plastics material such as high density polyethylene (HDPE). The body and base may be a different rigidity to the sealing element. Preferably, the body and base are more rigid than the sealing element.

The catheter assembly may comprise a fluid collection bag arranged to receive liquid from the distal end of the catheter. The catheter assembly may therefore be a closed catheter assembly in that liquid released from the bladder is collected by the fluid collection bag. The fluid collection bag may comprise two panels joined about their periphery. The fluid collection bag may be any suitable shape or size. The fluid collection bag may be rectangular. The fluid collection bag may form a volume capable of storing 700-1000 ml of liquid.

The catheter may comprise a funnel arranged at the distal end of the catheter. The funnel may be attached to the fluid collection bag. The funnel may arranged within the fluid collection bag. A fluid-tight seal may be provided between the funnel and the fluid collection bag. The funnel may be configured to deliver liquid from the distal end of the catheter into the fluid collection bag. The sleeve may be attached to the funnel. A fluid-tight seal may be provided between the sleeve and the funnel. The funnel may comprise bypass tubes to provide a fluid connection between the sleeve and fluid collection bag. Thus, liquid may efficiently pass from the catheter and/or sleeve into the fluid collection bag without leaking outside the catheter assembly.

The sleeve may comprise a flexible plastics material. The sleeve may be liquid impermeable. The sleeve may comprise a thermoplastic polyurethane (TPU) or low-density polyethylene (LDPE). Thus, the sleeve is cheap and easy to produce and can be easily manipulated by the user during use.

The catheter may be formed of a material of the group comprising: polyvinyl chloride, polytetrafluoroethylene, polyolefins, latex, silicones, synthetic rubbers, polyurethanes, polyesters, polyacrylates, polyamides, thermoplastic elastomeric materials, styrene block copolymers, polyether block amide, thermoplastic vulcanizates, thermoplastic copolyesters, thermoplastic polyamides, and water disintegrable or enzymatically hydrolysable material, or combinations, blends or co-polymers of any of the above materials.

The water disintegrable or enzymatically hydrolysable material may comprise a material of the group comprising: polyvinyl alcohol, extrudable polyvinyl alcohol, polyacrylic acids, polylactic acid, polyesters, polyglycolide, polyglycolic acid, poly lactic-co-glycolic acid, polylactide, amines, polyacrylamides, poly(N-(2-Hydroxypropyl) methacrylamide), starch, modified starches or derivatives, amylopectin, pectin, xanthan, scleroglucan, dextrin, chitosans, chitins, agar, alginate, carrageenans, laminarin, saccharides, polysaccharides, sucrose, polyethylene oxide, polypropylene oxide, acrylics, polyacrylic acid blends, poly (methacrylic acid), polystyrene sulfonate, polyethylene sulfonate, lignin sulfonate, polymethacrylamides, copolymers of aminoalkyl-acrylamides and methacrylamides, melamine-formaldehyde copolymers, vinyl alcohol copolymers, cellulose ethers, poly-ethers, polyethylene oxide, blends of polyethylene-polypropylene glycol, carboxymethyl cellulose, guar gum, locust bean gum, hydroxypropyl cellulose, vinylpyrrolidone polymers and copolymers, polyvinyl pyrrolidone-ethylene-vinyl acetate, polyvinyl pyrrolidone-carboxymethyl cellulose, carboxymethyl cellulose shellac, copolymers of vinylpyrrolidone with vinyl acetate, hydroxyethyl cellulose, gelatin, poly-caprolactone, poly(p-dioxanone), or combinations, blends or co-polymers of any of the above materials.

Preferably, the catheter is formed of a polyolefin material, in particular polyethylene and/or polypropylene.

Preferably, the catheter is formed of a thermoplastic elastomeric material.

The catheter may be a urinary catheter. The catheter may be a male urinary catheter. The catheter may be a female urinary catheter. The catheter may be an intermittent catheter. In one embodiment, the catheter is an intermittent male urinary catheter. Thus, the features of the present invention allow intermittent male urinary catheters to be adequately wetted prior to use which can be more difficult than for other types of catheter which are generally shorter.

The fluid reservoir may comprise an opening configured to allow fluid to exit the fluid reservoir to wet the catheter when the wetting mechanism is in the second configuration. The fluid reservoir may comprise a sealing element configured to provide a fluid-tight seal between the opening and the base when the wetting mechanism is in the first configuration. The sealing element may be resiliently deformed by compression between the base and body. The sealing element may inhibit inadvertent rotation of the base with respect to the body.

According to a broad aspect of the invention there is provided a catheter assembly comprising: a catheter comprising a proximal end for insertion into the body and a distal end, and a wetting mechanism, wherein the wetting mechanism comprises a base and a body, the body comprising a fluid reservoir, the base and body being rotatable with respect to one another to define a first configuration and a second configuration of the wetting mechanism, wherein the fluid reservoir comprises an opening configured to allow fluid to exit the fluid reservoir to wet the catheter when the wetting mechanism is in the second configuration, the fluid reservoir comprises a sealing element configured to provide a fluid-tight seal between the opening and the base when the wetting mechanism is in the first configuration and the sealing element is resiliently deformed by compression between the base and body. The sealing element may inhibit inadvertent rotation of the base with respect to the body.

According to a second aspect of the invention there is provided a catheter assembly comprising: a catheter comprising a proximal end for insertion into the body and a distal end, and a wetting mechanism, wherein the wetting mechanism comprises a base and a body, the body comprising a fluid reservoir, the base and body being rotatable with respect to one another to define a first configuration and a second configuration of the wetting mechanism, wherein the fluid reservoir comprises an opening configured to allow fluid to exit the fluid reservoir to wet the catheter when the wetting mechanism is in the second configuration, the fluid reservoir comprises a sealing element configured to provide a fluid-tight seal between the opening and the base when the wetting mechanism is in the first configuration and the sealing element is resiliently deformed by compression between the base and body and inhibits inadvertent rotation of the base with respect to the body.

Advantageously, the wetting mechanism is easier and more intuitive to use as the user can simply rotate the base and body with respect to one another to release wetting fluid from the fluid reservoir. This is an easier and simpler task that can be done in a more controlled manner even for users with reduced manual dexterity. In addition, the sealing element ensures that the wetting fluid is not prematurely released from the fluid reservoir and also helps the user to better control the fluid release process due to the resistance provided to rotation of the base with respect to the body. This increases the likelihood that the user will use the wetting mechanism as intended and that the wetting mechanism is not activated prematurely which could render the catheter unsafe to use. Furthermore, as the sealing element is simply compressed between the base and body to form a seal therebetween the wetting mechanism is simple and efficient to manufacture.

The catheter assembly may comprise a housing. The housing may comprises a/the base and a/the body. The base and body may be rotatable with respect to one another to define a first configuration and a second configuration of the housing. In the first configuration the housing may prevent passage of the proximal end of the catheter therethrough. In the second configuration the housing may permits passage of the proximal end of the catheter therethrough.

According to a third aspect of the invention there is provided a catheter assembly comprising: a catheter comprising a proximal end for insertion into the body and a distal end; and a housing, wherein the housing comprises a base and a body, the base and body are rotatable with respect to one another to define a first configuration and a second configuration of the housing, wherein in the first configuration the housing prevents passage of the proximal end of the catheter therethrough and in the second configuration the housing permits passage of the proximal end of the catheter therethrough.

Advantageously the housing is easier and more intuitive to use as the user can simply rotate the base and body with respect to one another to open the housing and allow the catheter to be accessed. This is an easier and simpler task that can be done in a more controlled manner even for users with reduced manual dexterity.

The housing may comprise any one or more of the features of the wetting mechanism as described in relation to the first and second aspects of the invention.

The catheter assemblies of the first to third aspects may include any one or more features of a catheter assembly as defined in general/broad terms, or according to any other of the first to third aspects set out above. The catheter assemblies of the first to third aspects may comprise any of the optional features of the others of the first to third aspects without necessarily including all the features required of them. That is to say, an optional feature which happens to be set out following one particular aspect does not necessarily apply only to that aspect, so, for example, the disclosure provides for a catheter assembly comprising: a catheter comprising a proximal end for insertion into the body and a distal end; and a housing, wherein the housing comprises a base and a body, the base and body are rotatable with respect to one another to define a first configuration and a second configuration of the housing, wherein in the first configuration the housing prevents passage of the proximal end of the catheter therethrough and in the second configuration the housing permits passage of the proximal end of the catheter therethrough as described in relation to the third aspect and wherein the body comprises a divider as described in relation to the first aspect.

According to a fourth aspect of the present invention there is provided a method of manufacturing a catheter assembly comprising the steps of providing a catheter comprising a proximal end for insertion into the body and a distal end, and a wetting mechanism comprising a base and a body, the body comprising a fluid reservoir, the base and body being rotatable with respect to one another to define a first configuration and a second configuration of the wetting mechanism, wherein in the second configuration the wetting mechanism permits release of fluid from the fluid reservoir to wet the catheter, and arranging the wetting mechanism at the proximal end of the catheter.

The method of the fourth aspect of the invention may be a method of manufacturing the catheter assembly of the first aspect of the invention, which, of course, may include any optional feature outlined above.

The method may comprise providing the adapter. The method may comprise providing the base. The method may comprise forming the sealing element, base and body independently of one another. The method may comprise forming the wetting mechanism by arranging the sealing element between the body and base. The method may comprise fitting the adapter onto the base. The method may comprise sealing the adapter to the base. The method may comprise aligning adapter tube co-axially with the base aperture/base guide tube. The method may comprise aligning the adapter wall and base wall so they are completely overlapping one another. The method may comprise moving the adapter in the axial direction towards the base. The method may comprise receiving at least one sealing rib inside the base.

The method may comprise providing the sealing element. The method may comprise providing the body. The method may comprise fitting the sealing element to the retainer such that retainer restricts movement of the sealing element with respect to the opening. The method may comprise attaching the body to the base. The method may comprise attaching the body to the base with the sealing element therebetween. The method may comprise inserting the axle into the locking aperture, for example until the locking protrusions engage the locking aperture. The method may comprise aligning the pin with the slot. The method may comprise receiving the pin in the slot.

The method may comprise providing a fluid-tight seal between the body and base. The method may comprise rotating the body with respect to the base until the opening is sealed by the sealing element. The method may comprise rotating the body with respect to the base until the opening corresponds to a flat section of the base wall. The method may comprise moving/rotating the pin to one end of the slot, preferably the end next to the base aperture/base guide tube. The method may comprise misaligning the body aperture/body guide tube and base aperture/base guide tube such that passage of the proximal end of the catheter therethrough is prevented. The method may comprise moving/rotating the body aperture/body guide tube to an opposite side of the wetting mechanism from the base aperture/base guide tube. The method may comprise aligning the outer edges of the base and body, for example so they are completely overlapping. The method may comprise moving/rotating the wetting mechanism into the first configuration.

The method may comprise providing wetting fluid. The method may comprise introducing wetting fluid into the fluid reservoir, for example into the body to fill the fluid reservoir.

The method may comprise attaching the inserter tip to the body. The method may comprise aligning the inserter tip with the body. The method may comprise aligning the insertion tube with the body aperture/body guide tube. The method may comprise fitting the sealing rib into the body to seal against the divider.

The method may comprise adding the cap over the inserter tip. The method may comprise sealing the fluid reservoir. The method may comprise sealing the through-hole with the plug.

The method may comprise arranging the proximal end of the catheter inside the adapter tube. The method may comprise arranging the sleeve around the catheter. The method may comprise attaching the sleeve to the outside of the adapter tube.

The method may comprise providing a funnel. The method may comprise providing a fluid collection bag. The method may comprise arranging the funnel within the fluid collection bag. The method may comprise providing a fluid-tight seal between the funnel and fluid collection bag. The method may comprise providing bypass tubes in the funnel to allow liquid in the sleeve to pass into the fluid collection bag.

According to a fifth aspect of the present invention there is provided a method of manufacturing a catheter assembly comprising providing a catheter comprising a proximal end for insertion into the body and a distal end, and a wetting mechanism, wherein the wetting mechanism comprises a base and a body, the body comprising a fluid reservoir, the base and body being rotatable with respect to one another to define a first configuration and a second configuration of the wetting mechanism, wherein the fluid reservoir comprises an opening configured to allow fluid to exit the fluid reservoir to wet the catheter when the wetting mechanism is in the second configuration, the fluid reservoir comprises a sealing element configured to provide a fluid-tight seal between the opening and the base when the wetting mechanism is in the first configuration and the sealing element is resiliently deformed by compression between the base and body and inhibits inadvertent rotation of the base with respect to the body.

The method of the fifth aspect of the invention may be a method of manufacturing the catheter assembly of the second aspect of the invention, which, of course, may include any optional feature outlined above.

The method may comprise arranging the wetting mechanism at the proximal end of the catheter.

According to a sixth aspect of the invention there is provided a method of manufacturing a catheter assembly comprising providing a catheter comprising a proximal end for insertion into the body and a distal end and a housing, wherein the housing comprises a base and a body, the base and body are rotatable with respect to one another to define a first configuration and a second configuration of the housing, wherein in the first configuration the housing prevents passage of the proximal end of the catheter therethrough and in the second configuration the housing permits passage of the proximal end of the catheter therethrough.

The method of the sixth aspect of the invention may be a method of manufacturing the catheter assembly of the third aspect of the invention, which, of course, may include any optional feature outlined above.

The method may comprise arranging the housing at the proximal end of the catheter.

According to a seventh aspect there is provided a method of wetting a catheter, the catheter comprising a proximal end for insertion into the body and a distal end, the method comprising providing a wetting mechanism in a first configuration, and arranging the wetting mechanism at the proximal end of the catheter, wherein the wetting mechanism comprising a base and a body, the body comprises a fluid reservoir, the base and body are rotatable with respect to one another to define the first configuration and a second configuration of the wetting mechanism, wherein in the second configuration the wetting mechanism permits release of fluid from the fluid reservoir into the sleeve to wet the catheter, wherein the method comprises moving the wetting mechanism from the first configuration to the second configuration.

The method of the seventh aspect of the invention may be a method of wetting a catheter from the catheter assembly of the first aspect of the invention, which, of course, may include any optional feature outlined above and may be manufactured according to the fourth aspect of the invention.

The method may comprise removing the cap from the inserter tip. The method may comprise grasping and pulling the pull-ring. The method may comprise disengaging the plug from the detent/through-hole.

The method may comprise rotating the body with respect to the base. The method may comprise overcoming a frictional force provided by the sealing element. The method may comprise aligning the opening/outlet with the outlet opening. The method may comprise allowing wetting fluid to flow out of the fluid reservoir. The method may comprise allowing wetting fluid to flow through the base and into the sleeve, optionally via the adapter and/or recessed portion. The method may comprise funnelling wetting fluid towards the adapter tube and catheter using the recessed portion.

The method may comprise continuing to rotate the body and base with respect to each other. The method may comprise moving the wetting mechanism through the third configuration. The method may comprise aligning the opening/outlet with the base aperture/base guide tube. The method may comprise allowing wetting fluid to flow into the base aperture/base guide tube. The method may comprise allowing wetting fluid to flow directly onto the catheter and/or into the sleeve.

The method may comprise contacting the pin against the slot protrusions. The method may comprise overcoming the slot protrusions.

The method may comprise retaining the pin at an end of the slot, preferably the end distal from the base aperture/base guide tube. The method may comprise aligning the opening/outlet with an end of the outlet opening, preferably distal from the base aperture/base guide tube. The method may comprise aligning the body aperture/body guide tube with the base aperture/base guide tube.

The method may comprise inserting the insertion tube into the urethra. The method may comprise progressively moving the proximal end of the catheter through the wetting mechanism, for example through the base and body. The method may comprise introducing the catheter into the body, preferably via the insertion tube. The method may comprise allowing fluid to pass from the body and into the fluid collection bag via the catheter. Consequently, the method may be a method of using a catheter assembly.

According to an eighth aspect there is provided a method of wetting a catheter comprising a proximal end for insertion into the body and a distal end, the method comprising providing a wetting mechanism in a first configuration, wherein the wetting mechanism comprises a base and a body, the body comprising a fluid reservoir, the base and body being rotatable with respect to one another to define the first configuration and a second configuration of the wetting mechanism, wherein the fluid reservoir comprises an opening configured to allow fluid to exit the fluid reservoir to wet the catheter when the wetting mechanism is in the second configuration, the fluid reservoir comprises a sealing element configured to provide a fluid-tight seal between the opening and the base when the wetting mechanism is in the first configuration and the sealing element is resiliently deformed by compression between the base and body and inhibits inadvertent rotation of the base with respect to the body, moving the wetting mechanism from the first configuration to the second configuration.

The method of the eighth aspect of the invention may be a method of wetting a catheter from the catheter assembly of the second aspect of the invention, which, of course, may include any optional feature outlined above and may be manufactured according to the fifth aspect of the invention.

The method may comprise introducing the catheter by its proximal end into the urethra. The method may comprise allowing fluid to pass from the body and into the fluid collection bag via the catheter and/or the sleeve. Consequently, the method may be a method of using a catheter assembly.

The wetting mechanism/housing may be provided in the first configuration.

According to a ninth aspect of the present invention there is provided a method of opening a housing containing a catheter, wherein the housing comprises a base and a body, the base and body are rotatable with respect to one another to define a first configuration and a second configuration of the housing, wherein in the first configuration the housing prevents passage of the proximal end of the catheter therethrough and in the second configuration the housing permits passage of the proximal end of the catheter therethrough, the method comprising providing the housing in the first configuration and moving the housing from the first configuration to the second configuration.

The method of the ninth aspect of the invention may be a method of wetting a catheter from the catheter assembly of the third aspect of the invention, which, of course, may include any optional feature outlined above and may be manufactured according to the sixth aspect of the invention.

In the first configuration a body aperture of the body may be misaligned with a base aperture of the base to prevent passage of the proximal end of the catheter therethrough. In the second configuration the body aperture may be aligned with the base aperture to permit passage of the proximal end of the catheter therethrough. The method may comprise aligning the body aperture with the base aperture to permit passage of the proximal end of the catheter therethrough.

According to a preferred embodiment there is provided a method of opening a housing containing a catheter, wherein the housing comprises a base and a body, the base and body are rotatable with respect to one another to define a first configuration and a second configuration of the housing, the method comprising providing the housing in the first configuration and moving the housing from the first configuration in which a body aperture of the body is misaligned with a base aperture of the base to prevent passage of the proximal end of the catheter therethrough, to a second configuration in which the body aperture is aligned with the base aperture to permit passage of the proximal end of the catheter therethrough.

The method may comprise introducing the catheter by its proximal end into the urethra. The method may comprise allowing fluid to pass from the body and into the fluid collection bag via the catheter and/or the sleeve. Consequently, the method may be a method of using a catheter assembly.

The methods of the fourth to ninth aspects of the present invention may of course individually include any one or more of the features, optional or otherwise, of one another and further may include any one or more of the features, optional or otherwise, of the first to third aspects of the present invention.

### DETAILED DESCRIPTION OF THE INVENTION

In order that the invention may be more clearly understood one or more embodiments thereof will now be described, by way of example only, with reference to the accompanying drawings, of which:

FIG. 1 is a side view of a catheter assembly with the wetting mechanism in the first configuration;

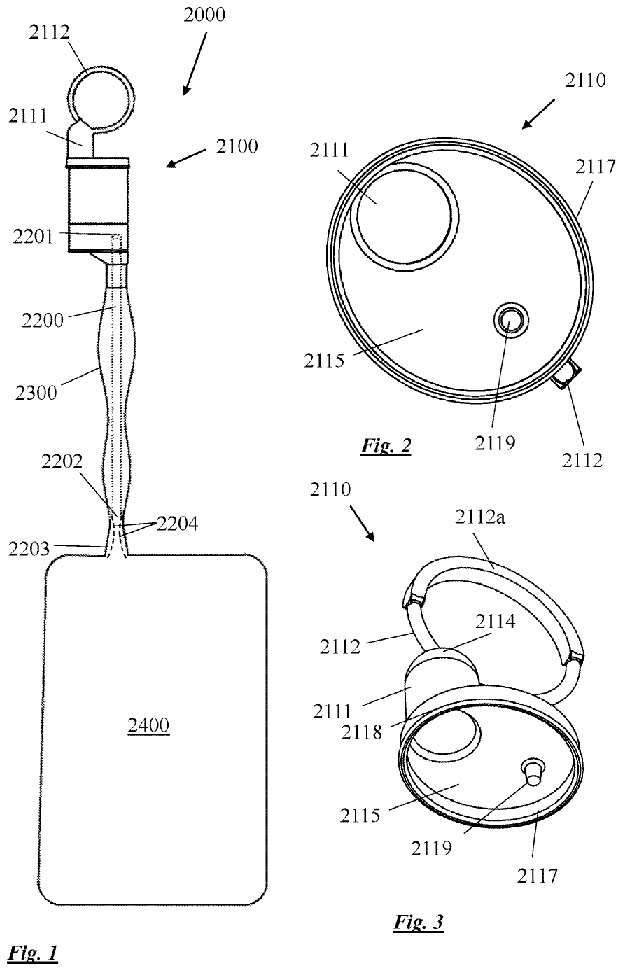

FIG. 2 is a bottom view of a cap of the catheter assembly of FIG. 1;

FIG. 3 is a bottom perspective view of the cap of FIG. 2;

FIG. 4 is a top view of an inserter tip of the catheter assembly of FIG. 1;

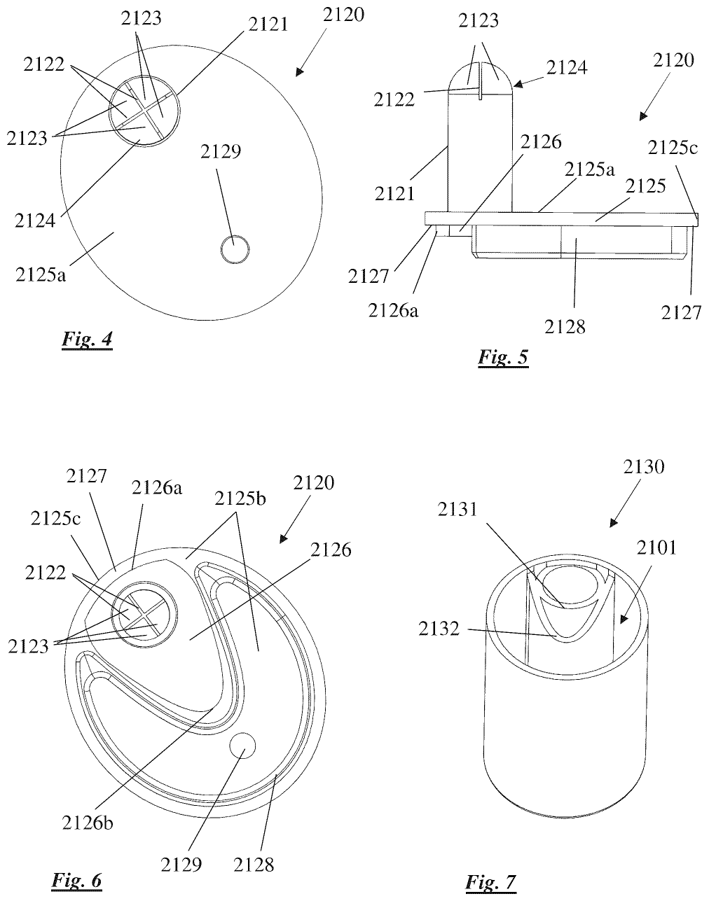

FIG. 5 is a side view of the inserter tip of FIG. 4;

FIG. 6 is a bottom view of the inserter tip of FIG. 4;

FIG. 7 is a top perspective view of a body of the catheter assembly of FIG. 1;

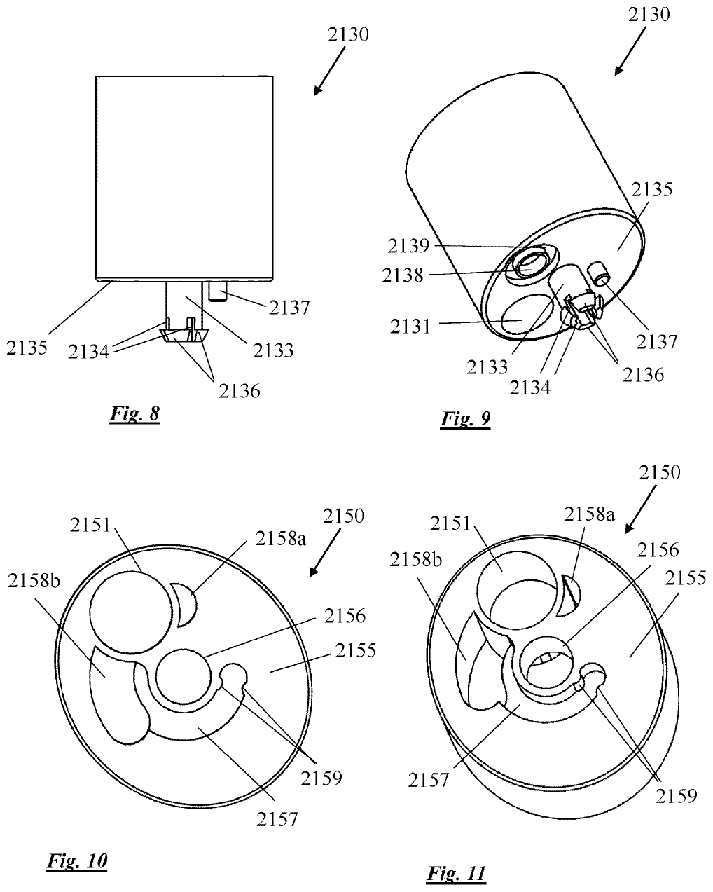

FIG. 8 is a side view of the body of FIG. 7;

FIG. 9 is a bottom perspective view of the body of FIG. 7;

FIG. 10 is a top view of a base of the catheter assembly of FIG. 1;

FIG. 11 is a top perspective view of the base of FIG. 10;

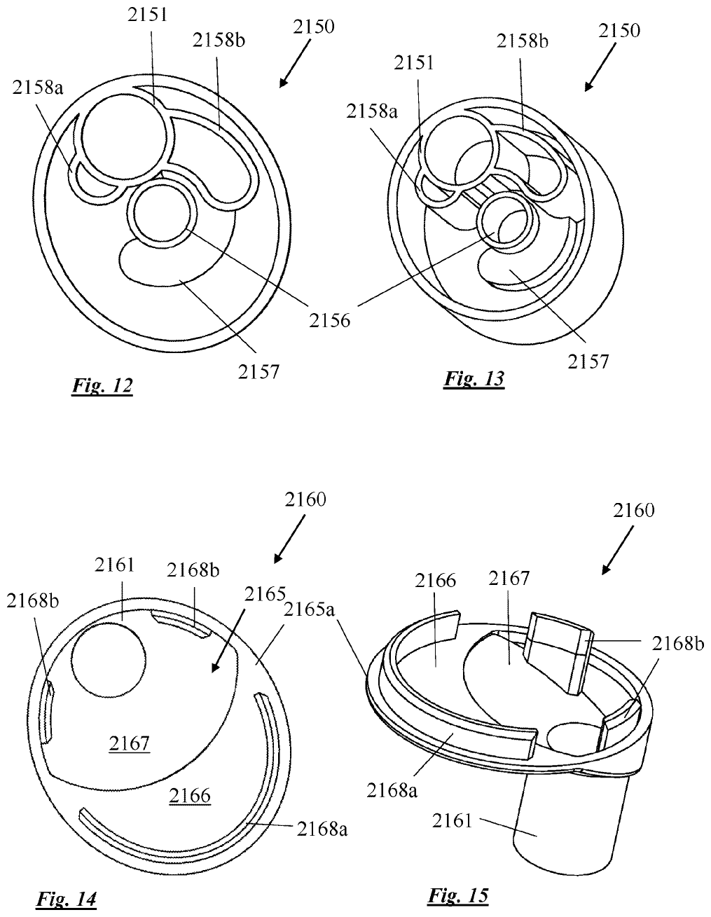

FIG. 12 is a bottom view of the base of FIG. 10;

FIG. 13 is a bottom perspective view of the base of FIG. 10;

FIG. 14 is a bottom view of an adapter of the catheter assembly of FIG. 1;

FIG. 15 is a bottom perspective view of the adapter of FIG. 14;

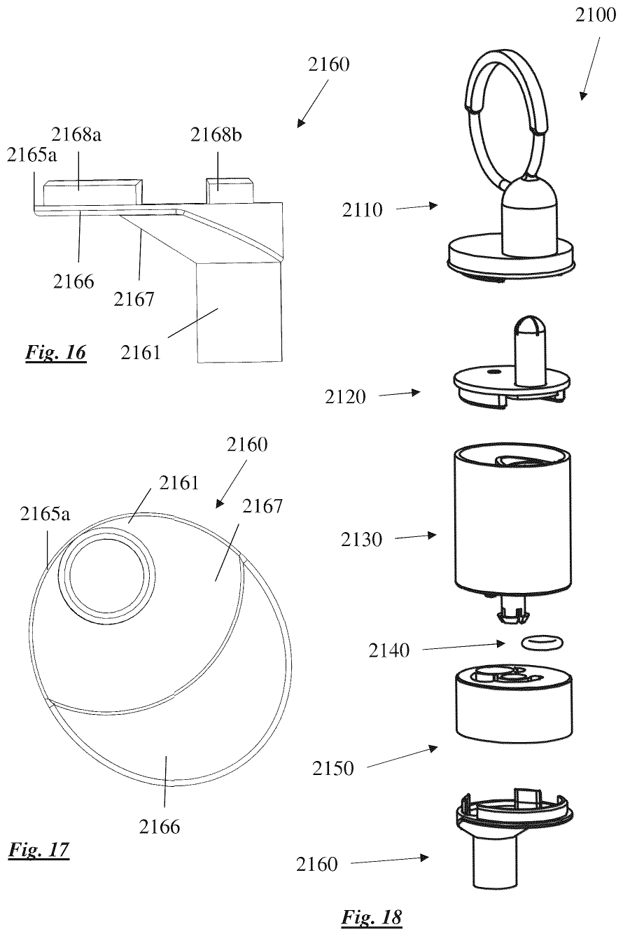

FIG. 16 is a side view of the adapter of FIG. 14;

FIG. 17 is a top view of the adapter of FIG. 14;

FIG. 18 is an exploded view of a wetting mechanism of the catheter assembly of FIG. 1;

FIG. 19 is an enlarged view of the wetting mechanism of the catheter assembly of FIG. 1 in the first configuration top perspective view of the catheter assembly of FIG. 1 during opening of the wetting mechanism;

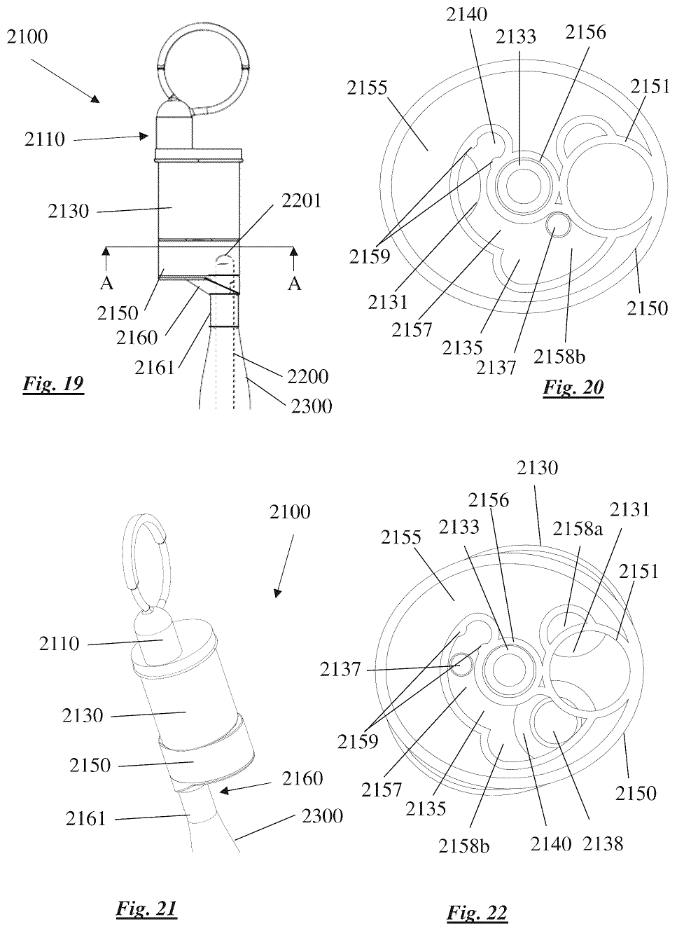

FIG. 20 is a cross-sectional view of the wetting mechanism of the catheter assembly of FIG. 1 in the first configuration;

FIG. 21 is a top perspective view of the catheter assembly of FIG. 1 with the wetting mechanism in the third configuration;

FIG. 22 is a cross-sectional view of the wetting mechanism of the catheter assembly of FIG. 1 in the third configuration;

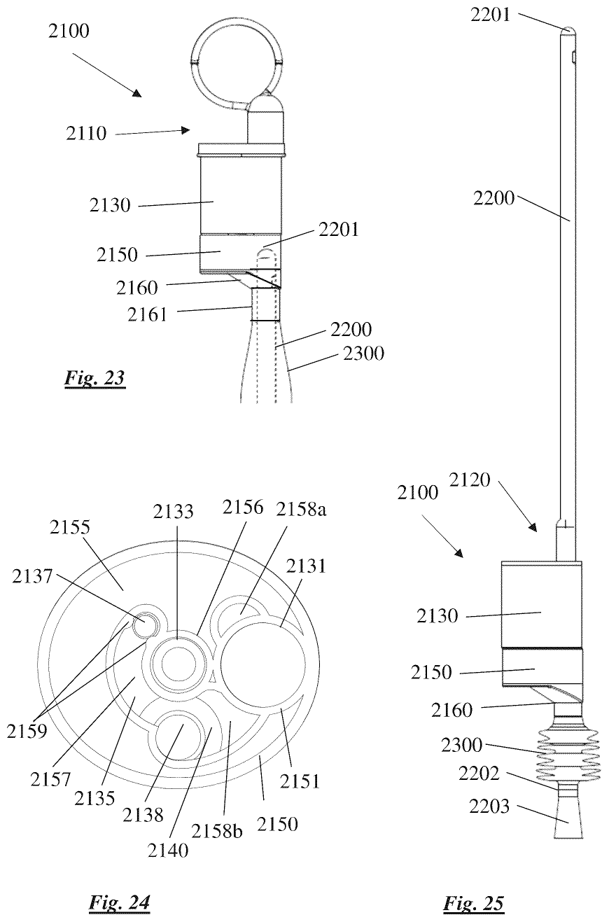

FIG. 23 is a side view of the catheter assembly of FIG. 1 with the wetting mechanism in the second configuration;

FIG. 24 is a cross-sectional view of the wetting mechanism of the catheter assembly of FIG. 1 in the second configuration; and

FIG. 25 is a side view of the catheter assembly of FIG. 1 with the catheter passing through the wetting mechanism.

Referring to FIGS. 1-25, in this embodiment a catheter assembly 2000 comprises a housing which in this example takes the form of a wetting mechanism 2100, a catheter 2200, a sleeve 2300 and a fluid collection bag 2400. The catheter 2200 comprises a proximal end 2201 for insertion into the user and a distal end 2202. In this embodiment, the catheter 2201 is a male urinary catheter made from a hydrophilic thermoplastic elastomer (TPE). The sleeve 2300 of this embodiment is a thermoplastic polyurethane (TPU) or low-density polyethylene (LDPE). The fluid collection bag 2400 is configured to receive liquid from the distal end of the catheter 2200 and comprises two panels joined about their peripheries to form a rectangular bag with a volume capable of storing 700-1000 ml of liquid. Obviously those skilled in the art will be able to select suitable alternative materials.

In this embodiment, the wetting mechanism 2100 is generally tubular and comprises a cap 2110, an inserter tip 2120, a body 2130, a sealing element 2140, a base 2150 and an adapter 2160 all independently formed and configured to fit together in an axial direction to form the wetting mechanism 2100, the axial direction defined along the axis of the tubular wetting mechanism 2100 from end to end. Consequently, each of cap 2110, inserter tip 2120, body 2130, base 2150 and adapter 2160 constitute a section along the length of the tubular wetting mechanism 2100.

In this embodiment, the wetting mechanism 2100 has an outer peripheral shape in a plane perpendicular to the axial direction defined by a cross-section. The cross-section has rotational symmetry of order 2, that is it is symmetric about a 180 degree rotation. The wetting mechanism has an elliptical cross-section defined by a minor axis and a major axis with vertices at either end of the major axis and co-vertices at either end of the minor axis as is conventional.

Of course, in other embodiments where the outer peripheral shape is not elliptical, a major axis and a minor axis may still be defined by the widest and narrowest points across the wetting mechanism respectively. In such other embodiments, the major and minor axis may not be orthogonal.

In this embodiment, the major axes of the inserter tip 2120, body 2130, base 2150 and adapter 2160 are all the same and 2-4 cm, for example 3.5 cm and their respective minor axes are approximately 10% less than their major axis. Of course these shapes/sizes are purely exemplary and others may be chosen or used as required/desired.

In this embodiment, the sealing element 2140 is formed of a flexible material such as a flexible plastics material, rubber or silicone-in this embodiment, the sealing element 2140 is an O-ring, preferably constructed from silicone. The cap 2110, inserter tip 2120, body 2130, base 2150 and adapter 2160 each comprise a material that is more rigid than the sealing element 2140 and preferably a different material all together, for example a hard plastics material such as high density polyethylene (HDPE).

In this embodiment the inserter tip 2120 comprises an insertion tube 2121 through which the proximal end 2201 of the catheter 2200 exits the wetting mechanism 2100 and passes into the user's body. The insertion tube 2121 is cylindrical with an inner diameter that is larger than the outer diameter of the catheter 2200, for example 20-30% of the major axis of the wetting mechanism 2100, for example 25% or 7 mm. The insertion tube 2121 has a length that is 50-60% of the major axis of the wetting mechanism 2100, for example 55%. The insertion tube 2121 has a constant thickness that is also the same as the wall thickness of the other parts of the inserter tip 2120 e.g. of 1 mm. The insertion tube 2121 is arranged parallel to the axial direction and is configured to be inserted into the urethra during use such that the catheter 2200 passes directly from the wetting mechanism 2100 into the user's body, this helps to reduce the risk of infection and discomfort as the catheter 2200 is guided smoothly into the body by the insertion tube 2121.

In this embodiment, the insertion tube 1121 is capped at one end with a hemicylindrical dome 2124 comprising two orthogonal slits 2122 arranged centrally on the dome 2124 to define four flaps 2123. The slits 2122 are configured to allow the flaps 2123 to separate as the proximal end 2201 of the catheter 2200 passes out through the dome 2124 from inside the insertion tube 2121. The domed shape also helps to facilitate comfortable insertion of the insertion tube 2121 into the body if required.

In this embodiment, the inserter tip 2120 comprises a tip wall 2125 that extends across the entire wetting mechanism 2100 perpendicular to the axial direction and helps to prevent over insertion of the insertion tube 2121 into the body while also sealing the inserter tip 2120 to the body 2130 as described below. The tip wall 2125 therefore has a shape that matches the cross-section of the wetting mechanism 2100 and a thickness of 5% of the length of the insertion tube 2121. The insertion tube 2121 extends out of a proximal surface 2125a of the tip wall 2125 and does not extend from the tip wall 2125 in the opposite direction, that is from an opposite distal surface 2125b of the tip wall 2125. In this embodiment, the insertion tube 2121 is arranged off-centre and specifically along the major axis of the elliptical tip wall 2125 approximately midway between a vertex and the centre of the tip wall 2125.

In this embodiment, the inserter tip 2120 further comprises a seat 2126 that extends from the distal surface 2125b of the tip wall 2125. The seat 2126 surrounds the insertion tube 2121 and provides a region of the tip wall 2125 that is 60-100% thicker than the rest, for example 80%, to assist with engagement of the inserter tip 2120 and body 2130 as described below. The seat 2126 also has a chamfered edge where it surrounds the insertion tube 2121 to assist with passage of the proximal end 2201 of the catheter 2200 into the insertion tube 2121.

In this embodiment, the seat 2126 has the general shape of an isosceles triangle with convex sides and rounded corners. A gap 2127 is provided between the base 2126a of the seat 2126 and an outer edge 2125c of the tip wall 2125 of approximately 3-5% of the major axis of the tip wall 2125, for example 4%. From either end of the base 2126a, the seat 2126 extends into and past the centre of the tip wall 2125. The seat 2126 has a height from the base 2126a to a tip vertex 2126b distal from the base 2126a of 55-60% of the major axis of the tip wall 2125, for example 57%. The seat 2126 has a width perpendicular to its height that is at a maximum approximately inline with the centre of the insertion tube 2121 along the major axis of the tip wall 2125. The seat 2125 width is 70-80% of the seat 2126 height, for example 75%.

In this embodiment, the inserter tip 2120 comprises a sealing rib 2128 that extends from the distal surface 2125b of the tip wall 2125. The sealing rib 2128 forms a continuous loop and follows the shape of the seat 2126 into the centre of the tip wall 2125 before extending around the perimeter of the tip wall 2125 that is not occupied by the seat 2125. The gap 2127 between the base 2126a of the seat 2126 and the edge 2125a of the tip wall 2125 is also maintained between the sealing rib 2128 and the edge 2125c of the tip wall 2125. The rib 2128 extends from the distal surface 2125b a distance of 2-3 times the thickness of the tip wall 2125, for example 2.5 times. The rib 2128 has a chamfered edge distal from the distal surface 2125b to assist with engagement between the sealing rib 2128 and body 2130 as described below.

In this embodiment, the inserter tip 2120 further comprises a detent in the form of a through-hole 2129 configured to engage the cap 2110 as described further below. The through-hole 2129 extends through the tip wall 2125 along the major axis and approximately midway between a vertex and the centre of the tip wall 2125 but on the other side from the insertion tube 2121. The through-hole 2129 is circular with a diameter of approximately half to one third the diameter of the insertion tube 2121.

In this embodiment, the cap 2110 is configured to cover the inserter tip 2120 and protect it before use. Consequently, the cap 2110 is a shell with the same shape as the inserter tip 2120 but a larger size such that it can efficiently enclose it and like numerals are used to denote similar features.

In this embodiment, the cap 2110 comprises a cap tube 2111 which is cylindrical with an inner diameter that matches the outer diameter of the insertion tube 2121. The cap tube 2111 is capped at one end with a hemicylindrical dome 2114 to fit over the dome 2124 of the inserter tip 2120.

In this embodiment, the cap 2110 comprises a cover 2115 extending out from the open end of the cap tube 2111 and configured to overlie the tip wall 2125. The cover 2115 therefore has an outer perimeter that is just outside the outer perimeter of the inserter tip 2120 and the body 2130, for example, the major axis of the cap 2110 may be 10% larger than the major axis of the inserter tip 2120 and body 2130.

In this embodiment, the cap 2110 comprises a cap flange 2117 that extends from the outer edge of the cover 2115 in the axial direction to overlie the inserter tip 2120 and body 2130. The cap flange 2117 has a length such that when the cap 2110 is placed over the inserter tip 2120, the cap flange 2117 extends down past the distal surface 2125b of the tip wall 2125 approximately 50-70% of the distance the sealing rib 2128 extends from the distal surface 2125b, for example 60%.

In this embodiment, at the free end of the cap flange 2117 a gripping protrusion 2118 extends around the outer perimeter of the cap flange 2117. The gripping protrusion 2118 occupies the bottom 20-30% of the cap flange 2117, for example the bottom 25% and has a semi-circular cross-section. In the region of the gripping protrusion 2118, the cap flange 2117 has a major axis that is 3% larger than the rest of the cap flange 2117. The gripping protrusion 2118 thereby helps ensure the cap 2110 remains attached to the wetting mechanism 2100 and also assists the user in removing the cap 2110 when required.

In this embodiment, the cap 2110 comprises a plug 2119 extending from the cover 2115 and configured to be received by the through-hole 2129 of the inserter tip 2110. The plug 2119 is shaped to be slightly larger than the through-hole 2129 where it meets the cover 2115 with a frustoconical shape such that it bears against the inside of the through-hole 2129 once received within it. This allows the plug 2119 to seal the through-hole 2129 and also to retain the cap 2110 on the inserter tip 2120.

In this embodiment, the cap 2110 further comprises a pull-ring 2112 attached to the dome 2114. The pull ring 2112 is configured to allow the user to grasp the cap 2110 and pull it off the wetting mechanism 2110. The pull-ring 2112 is arrange off-centre with respect to the cap tube 2111 such that the pull-ring 2112 is centrally located with respect to the axis of the wetting mechanism 2100. This helps the user to remove the cap 2110 as the position of the pull-ring 2112 reduces shearing forces that are not parallel to the axial direction which can cause the cap 2110 to remain stuck on the wetting mechanism 2100 due to the sides of the plug 211. Of course, in other embodiments, a different easily grippable feature may be used in place of the pull ring, such as a tab and the pull-ring or equivalent feature may be placed in a different position such as directly attached to the cover or cap flange.

In this embodiment, the pull-ring 2112 comprises a reinforced region 2112a. The reinforced region 2112a encompasses the half of the pull-ring 2112 distal from the cap 2110 and is strengthened to allow a greater force to be exerted onto the cap 2110 by the user. In this embodiment, the reinforced region 2112a has a widened square cross-section compared to the narrower circular cross-section of the rest of the pull-ring 2112.

In this embodiment the body 2130 is tubular with an elliptical cross-section of the same size as the inserter tip 2120, one closed end and an open end which is capped by the inserter tip 2120 in use as described below. The body 2130 has a length from end to end that is larger than its major axis, for example 3-10% larger, for example 5%. The outer tubular shell of the body 2130 has a thickness equivalent to the gap 2127 provided between the base 2126 of the seat 2126/sealing rib 2128 and the edge of the tip wall 2125c respectively such that the seat 2126 and sealing rib 2128 abut the body 2130 and seal therebetween.

In this embodiment, the body 2130 comprises a body aperture in the form of a body guide tube 2131 extending in the axial direction through the body 2130 and configured to ensure the proximal end 2201 of the catheter 2200 can pass smoothly through the body 2130 and into the insertion tube 2121. The guide tube 2131 is open-ended and cylindrical with a diameter that is larger than the diameter of the insertion tube 2121, for example 20-30% larger, for example 25%. The guide tube 2131 is also located co-axially with the insertion tube 2121 to ensure smooth passage of the catheter 2200 therethrough.

In this embodiment, the body 2130 comprises a divider 2132 that extends in the axial direction through the body 2130 around the guide tube 2131. The divider 2132 is arched and extends from outer perimeter of the body 2130 on either side of the guide tube 2131 and follows the shape that the sealing rib 2128 takes around the seat 2126. Thus, separating the guide tube 2131 from the remainder of the inside of the body 2130 and fluid reservoir 2101 as described below.

In this embodiment, the guide tube 2131 and divider 2132 extend from the closed end of the body 2130 approximately 95% of the way to the open end and thus leave a space for the seat 2126 to be accommodated inside the body 2130 when the wetting mechanism 2100 is put together as described below. Of course, in other embodiments a divider may have a different form perform the same function as the divider described herein. In some embodiments, the divider and guide tube may be integrated into the same feature that provides the functions of both the divider and guide tube.

In this embodiment, the body 2130 comprises a body wall 2135 that provides the closed end of the body 2130 as mentioned above. The guide tube 2131 extends from and through the body wall 2135 such that the catheter 2200 may pass into the body 2130 through the body wall 2135.

In this embodiment, the body wall 2135 comprises an axle 2133 configured to allow the body 2130 and base 2150 to rotate with respect to each about the axis defined by the axial direction. The axle 2133 has a length of 20-40% of the length of the body 2130, for example 30%, and is arranged at the centre of the elliptical body wall 2135 and extending away from the body 2130 in the axial direction. The axle 2133 is cylindrical with a diameter that is 15-25% the major axis of the body 2130, for example 20% and open ends.

In this embodiment, the axle 2133 comprises four slits arranged with equal separations around the circumference of the axle 2133 (e.g. with 90 degree separations). The slits 2134 each extend in the axial direction 35-40% of the length of the axle from the end distal from the body 2130, for example 37.5%. There are four locking protrusions 2136 extending around the circumference of the end of the axle 2133 between adjacent slits 2134. Each locking protrusion 2136 spans 15-25% of the length of the axle 2136, for example 20% from the end of the axle 2133. Each protrusion is wedge-shaped such that the effective diameter of the axle 2133 increases from linearly by 35-45%, for example 40% due to the locking protrusions 2136 that are narrowest at the tip of the axle 2133 distal from the body 2130. Thus, due to the presence of the slits 2134, each locking protrusion 2136 may flex inwardly as they are received by a locking aperture 2156 of the base 2150 and then flex back outwardly once inside the locking aperture 2156 to hold the body 2130 and base together 2140 as described further below.

In this embodiment, the wetting mechanism 2100 comprises a rotation guide in the form of two interlocking members: a pin 2137 and slot 2157. The body wall 2135 comprises the pin 2137 that extends in the axial direction from the body wall 2135 away from the body 2130. The pin 2137 is cylindrical with a capped end and a diameter of 5-10%, for example 7.5%, the major axis of the body wall 2135. The pin 2136 has a length of 30-40% the length of the axle 2133, for example 35%. The pin 2136 is configured to be received in the slot 2157 as described below.

In this embodiment, a fluid reservoir 2101 is defined by the inserter tip 2120, body 2130, divider 2132 and body wall 2135. The fluid reservoir 2101 is configured to hold wetting fluid within it and then release the wetting fluid when ready to activate the surface of the catheter 2200 ready for use, as described further below.

In this embodiment, to allow wetting fluid to leave the fluid reservoir 2101, the body wall 2135 comprises an opening in the form of an outlet 2138. The outlet 2138 is circular with a diameter of 60-70% of the diameter of the guide tube 2131, for example 65%.

In this embodiment, the body wall 2135 further comprises a retainer in the form of a circular sealing channel 2139 that is independent of the outlet 2138 but arranged concentrically around it. The sealing channel 2139 is sized to receive the sealing element 2140 such that the sealing element 2140 is compressed between the body 2130 and base 2150 to form a fluid tight seal around the outlet 2138 and between the base 2150 and body 2130 when they are joined together as described below.

In this embodiment, the guide tube 2131 is arranged between a vertex and the centre of the elliptical cross section of the body 2130 to align with the insertion tube 2121 of the inserter tip 2120. The outlet 2138 is positioned midway between a co-vertex and the centre of the cross-section of the body 2130, so that when viewing the body 2130 in the axial direction with the axle 2133 pointing towards the viewer, the outlet 2138 is positioned 90 degrees clockwise from the guide tube 2131 and about the axis of the body 2130. Due to the size of the sealing channel 2139, the sealing channel 2139 extends to a point outside the normal internal perimeter of the body 2130 and as such, the thickness of the outer shell of body 2130 adjacent the sealing channel 2139 is reduced slightly. The pin 2137 is positioned a further 140 degrees clockwise around the axis of the body 2130 from the outlet 2138. The pin 2137 is positioned adjacent to the axel 2133 and approximately 40% of the distance between the centre of the axle 2133 and the edge of the body 2130 as measured through the pin 2137. Of course, the exact positions of the guide tube 2131, outlet 2138 and pin 2137 may vary in other embodiments depending on the configuration of the base 2140 and body 2130.

In this embodiment, the base 2150 is tubular with an identical cross-section to the body 2130 but with a length that is 40-50% the length of the body 2130 for example 45%. The base 2150 has one end capped by a base wall 2155, and an opposite open end. The locking aperture 2156 mentioned above is arranged centrally in the base wall 2155 and is circular with a diameter just larger than the diameter of the axle 2133. In this embodiment, the locking aperture is tubular with a length the extends away from the base wall 2155 in the axial direction inside the base 2150 by a distance equivalent to the distance between the body wall 2135 and the locking protrusions 2136 of the axle 2133.

In this embodiment, the base 2150 comprises a base aperture in the form of a base guide tube 2151 that has a similar functionality as the body guide tube 2131. The base guide tube 2151 is the same size and shape as the body guide tube 2131 and both are arranged between a vertex and the centre of the cross section of the wetting mechanism 2100 such that the catheter 2200 may pass therethrough easily. The base guide tube 2151 extends from the base wall 2155 the full length of the base 2150 in the axial direction.

In this embodiment, base 2150 comprises an outlet opening 2158 configured to provide a fluid connection between the base 2150 and the fluid reservoir 2101 via the outlet 2158. The outlet opening 2158 comprises an arced opening in the base wall 2155 that extends through an arc equivalent to an angle of 150-160 degrees around the centre of base wall 2155, for example starting 40 degrees to one side of the guide tube 2151 and then ending 110 degrees to the other side (both times measured from the centre of the guide tube 2151). The outlet opening 2158 spans from a radius of 40% of the minor axis of the base wall 2155 to a radius of 60% of the minor axis of base wall 2155. The outlet opening 2158 is thereby aligned with the outlet 2138 when the body 2130 rotates with respect to the base 2150 as described below.

In this embodiment, where the guide tube 2151 corresponds to the position of the outlet opening 2158, the guide tube 2151 intersects the outlet opening 2158 and the volume contained by the guide tube 2151 is therefore cut off from the rest of the outlet opening 2158 to ensure that the catheter 2200 may not inadvertently leave the guide tube 2151 in use. This results in the outlet opening 2158 being formed from three separate portions, two portions either side of the guide tube 2151: a minor portion 2158a terminating at 40 degrees from the centre of the guide tube 2151 and a major portion 2158b terminating 110 degrees from the centre of the guide tube 2151, and the third portion being the guide tube 2151 itself. In this embodiment, the outlet opening 2158 is tubular and the edges of the outlet opening 2158 extend in the axial direction through the entire length of the base 2150 like the guide tube 2151. This helps to ensure the wetting fluid passes through the base 2150 to we the catheter 2200 efficiently.

In this embodiment, the slot 2157 mentioned above is arced in a similar fashion to the outlet opening 2158 and is configured to receive the pin 2127 to assist with rotation of the body 2130 and base 2150 with respect to one another. The slot 2157 has a depth that corresponds to the length of the pin 2137. The slot 2157 spans from a radius of 30% the minor axis of the base wall 2155 to a radius of 55% the minor axis of the base wall 2155. The slot 2157 covers an arc length equivalent to 210-220 degrees around the base wall 2155 starting from the edge of the guide tube 2151 corresponding to the major portion 2158b of the outlet element 2158 and extending around the base wall 2155 away from the guide tube 2151. Consequently, including slot 2157 permits the body 2130 and base 2150 to rotate up to 180 degrees with respect to one another. In the region where the major portion 2158b and slot 2157 are both present, the two are merged and form a continuous volume.

In this embodiment, a pair of slot protrusions 2159 are located at a position 30-40degrees in arc length around the base wall 2155 from the end of the slot 2157 distal from the guide tube 2151. The slot protrusions 2159 narrow the width of the slot 2157 to provide resistance to the movement of the pin 2137 past the slot protrusions 2159 and thus provide audible/tactile feedback to the corresponding rotation of the body 2130 with respect to the base 2150. In addition, as described above, the pin 2137 and slot protrusions 2159 inhibit rotation of the body 2130 with respect to the base 2150 and thus help the user to identify when the wetting mechanism 2100 is about to enter and leave the second configuration. In this embodiment, the torque required to move the pin 2137 past the slot protrusions 2159 is no more than 0.35 Nm.

In this embodiment, the base guide tube 2151, outlet opening 2158 and slot 2157 are all arranged such that when the body 2130 is attached to the base 2150 with the pin 2137 located at the end of the slot 2157 corresponding to the slot protrusions 2159, the body guide tube 2131 corresponds to the position of the base guide tube 2151 and the outlet 2158 corresponds to the end of the major portion 2158b of the outlet opening 2158.

In this embodiment, when the pin 2137 is located at the end of the slot 2157 distal from the slot protrusions 2159, the body guide tube 2131 and base guide tube 2151 are not aligned and passage of the catheter 2200 out of the base guide tube 2151 is blocked by the body wall 2135. Furthermore, the outlet 2158 is not aligned with the outlet opening 2158 and flow of wetting fluid out of the outlet 2158 is prevented by the sealing element 2140.

In this embodiment, the adapter 2160 is configured to provide a fluid connection between the base 2150 and sleeve 2300 as well as allowing the catheter 2200 to pass smoothly into the base guide tube 2151. The adapter 2160 comprises an adapter wall 2165 that has an outer edge 2165a that corresponds to the outer edge of the base 2150 and is configured to seal against the open end of the base 2150 as described below.

In this embodiment, the adapter wall 2165 comprises a flat portion 2166 and a recessed portion 2167 which extends away from the base 2150 in use. The flat portion 2166 is perpendicular to the axial direction and the edge 2165a of the adapter wall 2165 is also in the plane defined by the flat portion 2166. The flat portion 2166 and recessed portion 2167 are separated roughly along a line joining the co-vertices of the elliptical adapter wall 2165, except that the line is smoothly arced such that the flat portion 2166 encompasses one vertex and both co-vertices, whereas the recessed portion encompasses the other vertex and the central point of the adapter wall 2165.

In this embodiment, the adapter 2160 comprises an adapter tube 2161 attached to the recessed portion 2166 and providing access for fluids and the catheter 2200 through the adapter 2160 via the recessed portion 2166. The adapter tube 2161 is cylindrical and open-ended with a diameter slightly larger than the base guide tube 2151 diameter and 20-30% the major axis of the adapter wall 2165, for example 25%. The adapter tube 2161 is positioned such that the catheter 2200 can pass from the adapter tube 2161 into the base guide tube 2151 and is therefore positioned between the vertex encompassed by the recessed portion 2167 and the centre of the adapter wall 2165, for example 80% of the way between the two vertices of the adapter wall 2165.

In this embodiment, the adapter tube 2161 has a length of 45-55% of the major axis of the adapter wall 2165, for example 50%, and extends from the recessed portion 2167 away from the flat portion 2166. The recessed portion 2167 itself is recessed by 20-30% of the major axis of the adapter wall 2165, for example 25% and as such, a tip of the adapter tube 2161 distal from the flat portion 2166 is a distance from the flat portion equivalent to 65-85% of the major axis of the adapter wall 2165, for example 75%. From where the adapter tube 2161 extends from the recessed portion 2167, the recessed portion 2167 smoothly extends back to the flat portion 2166 and is consequently curved slightly due to the arced interface between the recessed and flat portions 2167, 2166 and the curved adapter tube 2161. Advantageously, this curved shape assists in funnelling wetting fluid from the outlet opening 2158 into the adapter tube 2161 as described below.

In this embodiment, the adapter 2160 comprises one major sealing rib 2168a and two minor sealing ribs 2168b which are configured to provide a fluid-tight seal between the base 2150 and adapter 2160 by engaging the base 2150 and preventing the base 2150 and adapter 2160 from separating. The major sealing rib 2168a and minor sealing ribs 2168b are therefore chamfered to provide a tight fit with the base 2150 as described below. The major sealing rib 2168a extends away from the flat portion 2166 in the opposite direct from the adapter 2160 to the adapter tube 2161 by a distance equivalent to 10% of the major axis of the adapter wall 2165. The major sealing rib 2168a is positioned just inside the edge 2165a of the adapter 2160 and extends around an arc of length equivalent to 160 degrees around the adapter wall 2165 and centred on the vertex encompassed by the flat portion 2166.

In this embodiment, the minor sealing ribs 2168b extend from the recessed portion 2167 such that they terminate at the same position as the major sealing rib 2168a as measured parallel to the axial direction. Each minor sealing rib 2168b is arc shaped and covers an arc length equivalent to 30 degrees around the adapter wall 2165. The minor sealing ribs 2168b are positioned each side of the vertex encompassed by the recessed portion 2167 and are separated from one another by an angular separation of 60 degrees around the adapter wall 2165 or 40 degrees from the corresponding edge of the major sealing rib 2168a.

In this embodiment, the wetting mechanism 2100 is constructed by first independently forming the different components, for example the base 2130, sealing element 2140 and body 2150 etc. are each independently formed. Then, arranging the cap 2110 and inserter tip 2120 co-axially on one side of the body 2130 and the base 2150 and adapter 2160 co-axially on the other side of the body 2130, with the sealing element 2140 in the channel 2139, and moving all parts of the wetting mechanism 2100 together in the axial direction as described below. Of course, this can be done in a variety of different ways and the method described below is purely exemplary.

In this embodiment, the adapter 2160 is fitted onto the base 2150. To do this, the adapter tube 2161 is aligned co-axially with the base guide tube 2151 such that the adapter wall 2165 and base wall 2155 are completely overlapping one another. The adapter 2160 is then moved in the axial direction towards the base 2150 so that the major sealing rib 2168a and minor sealing ribs 2168b are received inside the base 2150 and seal the adapter 2160 to the base 2150. Rotation of the adapter 2160 with respect to the base 2150 around an axis defined by the axial direction is inhibited by the sealing ribs 2168a, 2168b which will contact the base guide tube 2151 and outlet opening 2158 in the event of attempted rotation.

In this embodiment, the sealing element 2140 is then fitted into the channel 2139 of the body 2130 and the body 2130 attached to the base 2150. To do this, the axle 2133 is inserted into the locking aperture 2156 until the locking protrusions 2136 engage the locking aperture 2156 securing the body 2130 to the base 2150. In order for the axle 2133 to be sufficiently inserted into the locking aperture 2156, the pin 2137 must be aligned with the slot 2157 such that the pin 2137 is completely received in the slot 2157. Thus, the base 2150 and body 2130 both define different parts of the outer peripheral shape of the wetting mechanism, the base 2150 defines a part between the body 2130 and sleeve as described below.

In this embodiment, a fluid-tight seal between the body 2130 and base 2150 is created by rotating the body 2130 with respect to the base 2150 until the outlet 2138 corresponds to a flat section of the base wall 2155 and the sealing element 2140 is able to seal the outlet 2138. In this example, this corresponds to a position where the pin 2137 bears against one end of the slot 2157 and against the base guide tube 2151 and the body guide tube 2131 is on an opposite side of the major axis of the wetting mechanism 2100 from the base guide tube 2151. Once sealed, the outer edges of the base 2150 and body 2130 are completely aligned with one another such that the exterior of the wetting mechanism 2100 is continuous and smooth. The position of the base 2150 and body 2130 as described above defines a first configuration of the wetting mechanism 2100 where it is closed. The relative position of the base 2150 and body 2130 in the first configuration is shown best in FIG. 20 which is a cross-section through line A-A (see FIG. 19) when the wetting mechanism 2100 is in the first configuration.

In this embodiment, wetting fluid may now be introduced into the body 2130 to fill the fluid reservoir 2101. In this embodiment, the wetting fluid is water and interacts with the hydrophilic surface of the catheter 2200 to render it lubricious. In other embodiments, other wetting fluids may be used and they may be polar (e.g. water-based) or non-polar (e.g. oil-based) depending on the catheter's surface properties. In this embodiment, 12 ml of wetting fluid is placed inside the body 2130, of course in other embodiments more or less wetting fluid may be required.

In this embodiment, the inserter tip 2120 is then attached to the body 2130 by aligning the inserter tip 2120 with the body 2130 such that the insertion tube 2121 aligns with the body guide tube 2131 and the sealing rib 2128 fits into the body 2130 to seal against the divider 2132.

In this embodiment, the cap 2110 is then added over the inserter tip 2120 such that it seals the fluid reservoir 2101 via the plug 2119 sealing the through-hole 2129.

In this embodiment, the catheter 2200 is then arranged with the proximal end 2201 of the catheter 2200 just inside the adapter tube 2161. The sleeve 2300 is then arranged around the catheter 2200 and is attached to the outside of the adapter tube 2161 by any suitable means to form a fluid-tight seal between the sleeve 2300 and adapter 2160, for example a weld; mechanical seal; heat seal; pressure seal; adhesive; solvent bond; ultraviolet bond; ultrasonic weld; laser weld; impulse weld; or friction weld.

In this embodiment, the distal end 2202 of the catheter 2200 comprises a funnel 2203 and the funnel 2203 is arranged within the fluid collection bag 2400. A fluid-tight seal is provided between the funnel 2203 and fluid collection bag 2400 to inhibit leaks of fluid. A fluid-tight seal is also provided between the sleeve 2300 and funnel 2203 to ensure fluid cannot leak out of the sleeve 2300.

In this embodiment, the funnel 2203 comprises bypass tubes 2204 configured to allow liquid inside the sleeve 2300 to pass into the fluid collection bag 2400.

Referring to FIGS. 1 and 19-25, in this embodiment, to prepare the catheter 2200 for use, the user rotates the body 2130 with respect to the base 2150 to move the wetting mechanism 2100 from closed to open, that is from its first configuration to a second configuration. Advantageously, as the body is attached to the sleeve via the base, they can hold the base and sleeve together in one hand, then the longer body can be grasped with the other hand to rotate the body and base with respect to one another. To do this, the base 2150 and body 2130 must be rotated with respect to one another by 180 degrees and until the pin 2137 contacts and overcomes the slot protrusions 2159 and moves all the way to the end of the slot 2157 distal from the base guide tube 2151 as described below.

In this embodiment, before rotation of the base 2150 and body 2130, the cap 2110 is first removed from the inserter tip 2120 by grasping and pulling the pull-ring 2112. This disengages the plug 2119 from the through-hole 2129 which ensures that air may flow into the fluid reservoir 2101 to allow wetting fluid to leave the fluid reservoir 2101 via the outlet 2158 as described below.

In this embodiment, the body 2130 is then rotated with respect to the base 2150. Rotation is inhibited by the sealing element 2140 which is compressed between the base 2150 and body 2130 to provide friction between them which must be overcome by the user. This friction is advantageous as it inhibits inadvertent opening of the wetting mechanism 2100 prior to use. In this embodiment, the sealing element 2140 provides a frictional force that requires 0.35 Nm of torque about the edge of the wetting mechanism 2100 to overcome. Once the friction is overcome rotation may begin and after only 40 degrees of rotation the outlet 2138 becomes aligned with the minor portion 2158a of the outlet opening 2158 allowing wetting fluid to flow out of the fluid reservoir 2101, through the base 2150 and into the sleeve via the adapter 2160 and recessed portion 2167 that funnels wetting fluid towards the adapter tube 2161 and catheter 2200. Advantageously, as the proximal end 2201 of the catheter 2200 is positioned in the adapter tube 2161 this ensures the first part of the catheter 2200 to enter the body, that is the proximal end 2201, is the part most likely to be wetted by the wetting mechanism 2100. This helps reduce the likelihood of discomfort or injury during use.

In this embodiment, as the body 2130 and base 2150 are rotated further, the outlet 2138 then becomes aligned with the base guide tube 2151 and wetting fluid flows directly onto the proximal end 2201 of the catheter 2200 and into the sleeve 2300.

In this embodiment, further rotation aligns the outlet 2138 with the major portion 2158b of the outlet opening 2158 and eventually the pin 2137 contacts the slot protrusions 2159 as mentioned above. The user must then overcome the slot protrusions 2159 to fully open the wetting mechanism 2100 and move the wetting mechanism 2100 into the second configuration. This provides audible/tactile feedback as the pin 2137/locking protrusions 2159 are deformed slightly by this action. The relative position of the base 2150 and body 2130 in the second configuration is shown best in FIG. 24 which is a cross-section through line A-A (see FIG. 19) when the wetting mechanism 2100 is in the second configuration.

In this embodiment, a third configuration of the wetting mechanism 2100 is defined as the positions above wherein the outlet 2138 is in fluid communication with the outlet opening 2158 and the wetting mechanism 2100 is not in the second configuration, thus fluid can flow but the catheter is prevented from moving through the wetting mechanism 2100. The relative position of the base 2150 and body 2130 in the third configuration is shown best in FIG. 22 which is a cross-section through line A-A (see FIG. 19) when the wetting mechanism 2100 is in the third configuration.

In this embodiment, once in the opened, rotation out of the open position is inhibited by the slot protrusions 2159 which act to retain the pin 2137 at the end of the slot 2157 distal from the base guide tube 2151. The outlet 2138 is also aligned with one end of the major portion 2158b of the outlet opening 2158 to ensure all wetting fluid passes out of the fluid reservoir 2101 to wet the catheter 2200.

In this embodiment, in the second configuration the body guide tube 2131 and base guide tube 2151 are now also completely aligned, whereas in the first and third configurations they are not aligned. In the second configuration the adapter tube 2161, base guide tube 2151, body guide tube 2131 and insertion tube 2121 form a continuous bore through the wetting mechanism 2100 for the catheter 2200. The user may now insert the insertion tube 2121 into the urethra and then progressively move the proximal end 2201 of the catheter 2200 through the wetting mechanism 2100 and into the body via the insertion tube 2121 until urine flows from the bladder therethrough and into the fluid collection bag 2400.

The one or more embodiments are described above by way of example only. Many variations are possible without departing from the scope of protection afforded by the appended claims.

1. A catheter assembly comprising:
a catheter comprising a proximal end for insertion into the body and a distal end, and
a housing, wherein the housing comprises a base and a body, base and body are rotatable with respect to one another to define a first configuration and a second configuration of the housing,
wherein housing prevents passage of the proximal end of the catheter therethrough and in the second configuration the housing permits passage of the proximal end of the catheter therethrough.

2. A catheter assembly according to claim 1 wherein the catheter is an intermittent male urinary catheter.

3. A catheter assembly according to claim 1, wherein the base comprises a base aperture sized to allow passage of the catheter therethrough and wherein the body comprises a body aperture sized to allow passage of the catheter therethrough, in the first configuration the body aperture is misaligned with the base aperture to prevent passage of the proximal end of the catheter therethrough and in the second configuration body aperture is aligned with the base aperture to permit passage of the proximal end of the catheter therethrough.

4. A catheter assembly according to claim 3 wherein the body comprises a body guide tube and the base comprises a base guide tube, wherein the body aperture and base aperture are provided by the body guide tube and base guide tube respectively.

5. A catheter assembly according to claim 1 wherein the body comprises a fluid reservoir and the housing comprises a third configuration between the first and second configurations wherein, in the first configuration the housing inhibits release of fluid from the fluid reservoir to wet the catheter, in the third configuration the fluid reservoir is configured to release fluid into the sleeve to wet the catheter and prevent passage of the proximal end of the catheter therethrough.

6. A catheter assembly according to claim 5 wherein the base comprises a base aperture to allow passage of the catheter therethrough and an outlet opening, the outlet opening configured to direct wetting fluid onto the catheter to wet the catheter when the housing is in the third configuration, wherein the outlet opening is at least partially non-overlapping with a base aperture.

7. A catheter assembly according to claim 1 wherein the body comprises an axle configured to allow the body and base to rotate with respect to each other.

8. A catheter assembly according to claim 1 wherein the housing comprises a rotation guide configured to restrict linear movement of the base with respect to the body during rotation of the base with respect to the body.

9. A catheter assembly according to claim 8 wherein the rotation guide comprises at least two interlocking members.

10. A catheter assembly according to claim 9 wherein the at least two interlocking members comprise a pin and a slot.

11. A catheter assembly according to claim 10 wherein the body comprises the pin and the pin extends away from the body and the base comprises the slot configured to receive the pin.

12. A catheter assembly according to claim 10 wherein the slot comprises one or more slot protrusions configured to inhibit movement of the pin along the slot and past the slot protrusions wherein the slot protrusions are positioned to provide audible/tactile feedback as the wetting mechanism enters the second configuration.

13. A catheter assembly according to claim 12 wherein the slot protrusions extend into the slot in a plane parallel to the plane of rotation of the body with respect to the base.

14. A catheter assembly according to claim 12 wherein the slot protrusions are separated from an end of the slot by a distance equivalent to the diameter of the pin.

15. A method of opening a housing containing a catheter, wherein the housing comprises a base and a body, the base and body are rotatable with respect to one another to define a first configuration and a second configuration of the housing, wherein in the first configuration the housing prevents passage of the proximal end of the catheter therethrough and in the second configuration the housing permits passage of the proximal end of the catheter therethrough, the method comprising providing the housing in the first configuration and moving the housing from the first configuration to the second configuration.

16. A method according to claim 15, wherein in the first configuration a body aperture of the body is misaligned with a base aperture of the base to prevent passage of the proximal end of the catheter therethrough, the method further comprising aligning the body aperture with the base aperture to permit passage of the proximal end of the catheter therethrough.

17. A method according to claim 16 further comprising moving the catheter through the base aperture and into the body via the body aperture.
